(19) United States
(12) Patent Application Publication
(10) Pub. No.: US 2014/0186845 A1
(43) Pub. Date: Jul. 3, 2014

(54) METHODS FOR DETERMINING THE EXPRESSION LEVEL OF A GENE OF INTEREST INCLUDING CORRECTION OF RT-QPCR DATA FOR GENOMIC DNA-DERIVED SIGNALS

(71) Applicants: Henrik Laurell, Toulouse (FR); Mikael Kubista, Goteborg (SE); Jason Iacovoni, Toulouse (FR)

(72) Inventors: Henrik Laurell, Toulouse (FR); Mikael Kubista, Goteborg (SE); Jason Iacovoni, Toulouse (FR)

(73) Assignees: UNIVERSITE PAUL SABATIER - TOULOUSE III, Toulouse Cedex 9 (FR); INSERM (Institut National de la Sante et de la Recherche Medicale), Paris (FR)

(21) Appl. No.: 14/125,675

(22) PCT Filed: Jun. 14, 2012
(86) PCT No.: PCT/EP2012/061289

§ 371 Date: Feb. 11, 2014

Related U.S. Application Data
(60) Provisional application No. 61/496,675, filed on Jun. 14, 2011.

(30) Foreign Application Priority Data
Jan. 5, 2012 (EP) 12305014.8

(51) Int. Cl. C12Q 1/68

(52) U.S. Cl. CPC C12Q 1/6848
USPC 435/6.12; 536/24.33

(57) ABSTRACT

The present invention relates to methods for determining the expression level of a gene of interest in a nucleic acid sample by RT-qPCR. More specifically, procedures for determining the impact of a gDNA contamination on the measured total signal have been developed allowing the correction of the signal originating from the above said gDNA. A further aspect of the invention refers to a mean by which the sensitivity qPCR primers toward gDNA can be determined.

### FIELD OF THE INVENTION

The present invention relates to methods for determining the expression level of a gene of interest in a nucleic acid sample by PCR.

### BACKGROUND OF THE INVENTION

Accurate gene expression analysis by reverse transcription (RT) quantitative PCR (qPCR) requires assays with high specificity for the target cDNA/reference gene, collectively referred to herein as the Gene-Of-Interest (GOI). It is important to have negligible signal contribution from experimental artifacts, such as primer-dimers and contaminating genomic DNA (gDNA). Traditionally, primer-dimer formation is tested using a “no template control” (NTC) and gDNA contamination levels are measured with RT(−) controls [which differ from regular RT(+) reactions in that no reverse transcriptase is added]. Contamination of gDNA is an inherent problem during RNA purification due to the similar physicochemical properties of RNA and DNA. Since gDNA contamination levels are frequently not uniform between samples (Bustin, 2002) and the sensitivity toward gDNA differs greatly between GOI assays, RT(−) controls are needed for each sample/assay pair, which substantially adds to the cost and labor in RTqPCR profiling studies. A difference of at least five quantification cycles (Cq) between RT(+) and RT(−) reactions indicates that <3% of the total signal originates from gDNA, and is commonly used as limit to ensure accurate estimation of GOI expression. Smaller differences typically call for DNase treatment of samples.

The accuracy of gDNA background estimation, as measured with RT(−) reactions, is compromised due to the fact that GOI assays, designed to amplify target transcripts, are used even though they are not optimized for gDNA amplification. Furthermore, intrinsic characteristics of RT(−)qPCRs that influence the result of the correction, such as amplification efficiencies, are difficult to assess. In addition, as proposed theoretically (Peccoud and Jacob, 1996) and shown experimentally (Nordg\dot{a}rd et al., 2006; Bengtsson et al., 2008), a low initial number of target molecules leads to a large variability between replicates, mainly due to stochastic effects. All together, this explains the low reproducibility frequently observed in RT(−) reactions.

The qPCR assays can be either gDNA sensitive or insensitive. Whereas qPCR assays can be designed to be gDNA insensitive, such as those designed to target exons flanking a long intron or with primers that cross exonexon junctions, qPCR assays for single-exon genes will readily amplify contaminating gDNA. The gDNA background signal is even further amplified in the presence of multiple genomic copies or pseudogenes. The latter are particularly troublesome since they may originate from retrotransposons without introns that are amplified even with intron-spanning assays. Thus, there exists both variation in the degree of contamination between samples and large differences between assays in terms of their sensitivity to gDNA. Therefore, general methods of controlling and correcting for gDNA contamination are essential for accurate measurements of gene expression.

Accordingly there is a need in the art to methods that will allow the accurate determination of the expression level of a gene of interest in a RNA sample susceptible to genomic DNA contamination and thus that will allow avoiding use of DNAse pretreatment of the RNA sample.

As an alternative to RT(-) reactions, the inventors have developed a procedure that determines the impact of the gDNA contamination on the measured signal much more accurately and allows validation of qPCR primers with respect to their sensitivity toward gDNA. The inventors show in proof-of-principle experiments that efficient background correction can be performed with gDNA contamination representing 60% of the total signal.

### SUMMARY OF THE INVENTION:

The present invention relates to a method for determining the expression level of a gene of interest (GOI) in a nucleic acid sample by means of reverse transcription real-time PCR comprising the steps consisting of:
a) providing two aliquots of the nucleic acid sample
b) providing a pair of PCR primers specific for the gene of interest
c) treating a first aliquot with reverse transcriptase to produce complementary DNA (cDNA), performing a PCR on said aliquot with said pair of PCR primers of step b) and determining the Cq value (Cq_{RT(+)}^{GOI})
d) performing a PCR on a second aliquot of the nucleic acid sample with said pair of PCR primers of step b) and determining the Cq value (Cq_{RT(-)}^{GOI})
e) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{RT ( - )}^{GOI}} ) .$$

A further aspect of the invention relates to a method for determining the expression level of a gene of interest (GOI) in a nucleic acid sample, by means of reverse transcription real-time PCR comprising the steps consisting of:
a) providing a genomic DNA (gDNA) sample
b) providing a pair of PCR primers specific for the gene of interest
c) providing of at least one pair of PCR primers that specifically amplify a sequence present in at least one copy per haploid genome and that is not transcribed to any significant extent
d) treating said nucleic acid sample with reverse transcriptase to produce complementary DNA (cDNA)
e) performing a quantitative PCR on said nucleic acid sample of step d) with said pair of PCR primers of step b) and determining the Cq value (Cq_{RT(+)}^{GOI})
f) performing a quantitative PCR on said nucleic acid sample of step d) with said pair of PCR primers of step c) and determining the Cq value (Cq_{Sample}^{ValidPrime})
g) performing a quantitative PCR on the genomic DNA (gDNA) sample with said pair of PCR primers of step b) and determining the Cq value (Cq_{gDNA}^{GOI})
h) performing a quantitative PCR on the genomic DNA (gDNA) sample with the pair of PCR primers of step c) and determining the Cq value (Cq_{gDNA}^{ValidPrime})
i) calculating the Cq_{DNA}^{GOI} with the formula Cq_{DNA}^{GOI}=Cq_{gDNA}^{GOI}+(Ca_{Sample}^{ValidPrime}−Cq_{gDNA}^{ValidPrime})
j) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{DNA}^{GOI}} )$$

A further aspect of the invention relates to a method for determining the expression level of a gene of interest (GOI) in a nucleic acid sample, by means of reverse transcription real-time PCR comprising the steps consisting of:
a) providing two aliquots of the nucleic acid sample
b) providing two aliquots of a genomic DNA (gDNA) sample
c) providing a pair of PCR primers specific for the gene of interest
d) providing of at least one pair of PCR primers that specifically amplify a sequence present in at least one copy per haploid genome and that is not transcribed to any significant extent
e) treating a first aliquot of the nucleic acid sample with reverse transcriptase to produce complementary DNA (cDNA), performing a quantitative PCR on said aliquot with said pair of PCR primers of step c) and determining the Cq value (Cq_{RT(+)}^{GOI})
f) performing a quantitative PCR on the second aliquot of the nucleic acid sample with said pair of PCR primers of step d) and determining the Cq value (Cq_{Sample}^{ValidPrime})
g) performing a quantitative PCR on the first aliquot of the genomic DNA (gDNA) sample with said pair of PCR primers of step c) and determining the Cq value (Cq_{gDNA}^{GOI})
h) performing a quantitative PCR on the second aliquot of the genomic DNA (gDNA) sample with the pair of PCR primers of step d) and determining the Cq value (Cq_{gDNA}^{ValidPrime})
i) calculating the Cq_{RT(-)}^{GOI} with the formula Cq_{RT(-)}^{GOI}−Cq_{gDNA}^{GOI}+(Ca_{Sample}^{ValidPrime}−Cq_{gDNA}^{ValidPrime})
j) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{RT ( - )}^{GOI}} )$$

### DETAILED DESCRIPTION OF THE INVENTION

The inventors claim that DNase treatment is unnecessary for the majority of qPCR studies in eukaryotes, since it is possible to design gDNA-insensitive assays for most genes. Apart from adding extra costs to the protocol, the addition of the enzyme, buffers or added compounds, such as EDTA, can influence the performance of the qPCR. Incomplete DNAse inactivation can result in cDNA degradation post RT. For these reasons, and also since gDNA removal in small sample preparations is difficult, the inventors have developed different method that measure gDNA contamination and evaluate its impact on the observed signal.

Accordingly the present invention relates to a method for determining the expression level of a gene of interest (GOI) in a nucleic acid sample by means of reverse transcription real-time PCR comprising the steps consisting of:
a) providing two aliquots of the nucleic acid sample
b) providing a pair of PCR primers specific for the gene of interest
c) treating a first aliquot with reverse transcriptase to produce complementary DNA (cDNA), performing a PCR on said aliquot with said pair of PCR primers of step b) and determining the Cq value (Cq_{RT(+)}^{GOI})
d) performing a PCR on a second aliquot of the nucleic acid sample with said pair of PCR primers of step b) and determining the Cq value (Cq_{RT(-)}^{GOI})
e) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{RT ( - )}^{GOI}} ) .$$

As used herein the “nucleic acid sample” refers to a RNA sample susceptible to genomic DNA contamination. Typically, said nucleic acid sample is prepared form a mRNA preparation. The method of RNA preparation can be any method of RNA preparation that produces enzymatically manipulatable mRNA. For example, the RNA can be isolated by using the guanidinium isothiocyanate-ultracentrifugation method, the guanidinium and phenol-chloroform method, the lithium chloride-SDS-urea method or poly A+/mRNA from tissue lysates using oligo (dT) cellulose method (See for example, Schildkraut, C. L. et al., (1962) J. Mol. Biol. 4,430-433 ; Chomczynski, P. and Sacchi, N. Anal. Biochem. 162,156 (1987); Auffray and F. Rougeon (1980), Eur J Biochem 107: 303-314; Aviv H, Leder P. (1972), Proc Natl Acad Sci USA 69, 1408- 1412; Sambrook J, et al. , (1989). Selection of poly A+ RNA, In Molecular Cloning: vol. 1, 7.26-7. 29. All of which are herein incorporated by reference at least for material related to RNA purification and isolation) (WO2003/048377).

It is important when isolating the RNA that enough RNA is isolated. Furthermore, typically the quantity of RNA obtained can be determined. For example, typically at least 0.01 ng or 0.5 ng or 1 ng or 10 ng or 100 ng or 1,000 ng or 10,000 ng or 100,000 ng of RNA can be isolated (WO2003/048377).

The RNA can be isolated from any desired cell or cell type and from any organism, including mammals, such as mouse, rat, rabbit, dog, cat, monkey, and human, as well as other non-mammalian animals, such as fish or amphibians, as well as plants and even prokaryotes, such as bacteria. Thus, the DNA used in the method can also be from any organism, such as that disclosed for RNA.

In a particular embodiment, the nucleic acid sample may be treated with a DNAse, preferably a DNAse that is specific for double stranded DNA (e.g. shrimp DNAse).

As used herein a “pair of PCR primers”, also referred to qPCR assay, consists of a forward amplification primer and a reverse amplification primer. As used herein, “forward amplification primer” refers to a polynucleotide used for PCR amplification that is complementary to the sense strand of the target nucleic acid. “Reverse amplification primer” refers to a polynucleotide used for PCR amplification that is complementary to the antisense strand of the target nucleic acid. For a given target, a forward and reverse amplification primer are used to amplify the DNA in PCR. In a particular embodiment, the pair of PCR primes shall allow a PCR amplification efficiency of at least 90%.

Any reverse transcriptase well known in the art may be used according to the invention. Examples for reverse transcriptases include but are not limited to AMV Reverse Transcriptase (Roche Applied Science Cat. No. 11 495 062), MMuLV Reverse Transcriptase (Roche Applied Science Cat No. 011 062 603), and the recombinant Transcriptor Reverse Transcriptase (Roche Applied Science Cat. No. 03 531 317).

The PCR can be performed using any conditions appropriate for the primer pairs samples being used. Typically, the PCR mixture is heated for a period of time at a high temperature, such as 95 degrees C. The period of time can vary, but in general the time will be long enough to destroy any residual non-thermal stable polymerases which may be present in the mixture, for example, at least 5 minutes or 10 minutes or 15 minutes at 95 degrees C. The concentration of the dNTPs or primers or enzyme or buffer conditions can be any concentration that allows the PCR to occur. Typically the concentration of the dNTPs can be between 0.2 and 0.5 mM each. Typically the concentration of the primers can be between 0.1 and 0.5 μM each, however, typically the primer pairs will be at about the same concentration, i. e. equimolar. Typically the concentration of the enzyme can be between 1 to 3 units per reaction. Typically the concentration and make up of the buffers is for example 1× final concentration out of a 10× stock solution as suggested by the manufacturer of the thermal polymerase. But it is understood that conditions other than these can also work, and in some cases may be determined after empirical testing. Any type of thermal stable polymerase can be used.

In one major embodiment, the PCR performed on the aliquot is monitored in real time. Different detection formats are known in the art. The below mentioned detection formats have been proven to be useful for RT-PCR and thus provide an easy and straight forward possibility for gene expression analysis:

TaqMan Hydrolysis Probe Format: A single-stranded Hybridization Probe is labeled with two components. When the first component is excited with light of a suitable wavelength, the absorbed energy is transferred to the second component, the so-called quencher, according to the principle of fluorescence resonance energy transfer. During the annealing step of the PCR reaction, the hybridization probe binds to the target DNA and is degraded by the 5′-3′ exonuclease activity of the Taq Polymerase during the subsequent elongation phase. As a result the excited fluorescent component and the quencher are spatially separated from one another and thus a fluorescence emission of the first component can be measured. TaqMan probe assays are disclosed in detail in U.S. Pat. No. 5,210,015, U.S. Pat. No. 5,538,848, and U.S. Pat. No. 5,487,972. TaqMan hybridization probes and reagent mixtures are disclosed in U.S. Pat. No. 5,804,375.

SybrGreen Format: It is also within the scope of the invention, if real time PCR is performed in the presence of an additive according to the invention in case the amplification product is detected using a double stranded nucleic acid binding moiety. For example, the respective amplification product can also be detected according to the invention by a fluorescent DNA binding dye which emits a corresponding fluorescence signal upon interaction with the double-stranded nucleic acid after excitation with light of a suitable wavelength. The dyes SybrGreenl and SybrGold (Molecular Probes) or EvaGreen (Biotium) have proven to be particularly suitable for this application. Intercalating dyes can alternatively be used. However, for this format, in order to discriminate the different amplification products, it is necessary to perform a respective melting curve analysis (U.S. Pat. No. 6,174,670).

As used herein, “Cq” refers to quantification cycle values calculated from the record fluorescence measurements of the real time quantitative PCR. “Cq” refers to the number of cycles required for the PCR signal to reach the significant threshold. The calculated Cq value is proportional to the number of target copies present in the sample. The Cq quantification is performed with any method for the real time quantitative PCR amplification described in the art (Bustin, 2000 J Mol Endocrinol 25, 169-193; Gibson et al., 1996 Genome Res. 6,995-1001; Pabinger et al., 2009 BMC Bioinformatics. 10:268).

A further aspect of the invention relates to a method for determining the expression level of a gene of interest (GOI) in a nucleic acid sample by means of reverse transcription real-time PCR comprising the steps consisting of:
a) providing a genomic DNA (gDNA) sample
b) providing a pair of PCR primers specific for the gene of interest
c) providing of at least one pair of PCR primers that specifically amplify a sequence present in at least one copy per haploid genome and that is not transcribed to any significant extent
d) treating said nucleic acid sample with reverse transcriptase to produce complementary DNA (cDNA)
e) performing a quantitative PCR on said nucleic acid sample of step d) with said pair of PCR primers of step b) and determining the Cq value (Cq_{RT(+)}^{GOI})
f) performing a quantitative PCR on said nucleic acid sample of step d) with said pair of PCR primers of step c) and determining the Cq value (Cq_{Sample}^{ValidPrime})
g) performing a quantitative PCR on the genomic DNA (gDNA) sample with said pair of PCR primers of step b) and determining the Cq value (Cq_{gDNA}^{GOI})
h) performing a quantitative PCR on the genomic DNA (gDNA) sample with the pair of PCR primers of step c) and determining the Cq value (Cq_{gDNA}^{ValidPrime})
i) calculating the Cq_{DNA}^{GOI} with the formula Cq_{DNA}^{GOI}=Cq_{gDNA}^{GOI}+(Cq_{Sample}^{ValidPrime}−Cq_{gDNA}^{ValidPrime})
j) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{DNA}^{GOI}} )$$

A further aspect of the invention relates to a method for determining the expression level of a gene of interest (GOI) in a nucleic acid sample by means of reverse transcription real-time PCR comprising the steps consisting of:
a) providing two aliquots of the nucleic acid sample
b) providing two aliquots of a genomic DNA (gDNA) sample
c) providing a pair of PCR primers specific for the gene of interest
d) providing of at least one pair of PCR primers that specifically amplify a sequence present in at least one copy per haploid genome and that is not transcribed to any significant extent
e) treating a first aliquot of the nucleic acid sample with reverse transcriptase to produce complementary DNA (cDNA), performing a quantitative PCR on said aliquot with said pair of PCR primers of step c) and determining the Cq value (Cq_{RT(+)}^{GOI})
f) performing a quantitative PCR on the second aliquot of the nucleic acid sample with said pair of PCR primers of step d) and determining the Cq value (Cq_{Sample}^{ValidPrime})
g) performing a quantitative PCR on the first aliquot of the genomic DNA (gDNA) sample with said pair of PCR primers of step c) and determining the Cq value (Cq_{gDNA}^{GOI})
h) performing a quantitative PCR on the second aliquot of the genomic DNA (gDNA) sample with the pair of PCR primers of step d) and determining the Cq value (Cq_{gDNA}^{ValidPrime})
i) calculating the Cq_{RT(-)}^{GOI} (or Cq_{DNA}^{GOI}) with the formula Cq_{RT(-)}^{GOI}=Cq_{gDNA}^{GOI}+(Cq_{Sample}^{ValidPrime}−Cq_{gDNA}^{ValidPrime})
j) determining the expression level of said gene of interest (GOI) (Cq_{RNA}^{GOI}) with the formula

$$C {q}_{RNA}^{GOI} = - {log}_{2} ( {2}^{- {Cq}_{RT ( + )}^{GOI}} - {2}^{- {Cq}_{RT ( - )}^{GOI}} )$$

In one embodiment of the invention, the quantitative PCR of step f) is performed on the second aliquot of the nucleic acid sample that was previously reverse transcribed using reverse transcriptase to produce complementary DNA (cDNA).

As used herein, “genomic DNA sample” or “gDNA” refers to a genomic DNA sample prepared from a DNA preparation. Methods for DNA purification are well known in the art. The genomic DNA may be prepared from a cell that is of the same organism than the cell that is used for preparing the nucleic acid sample of the invention (e.g. Human or mouse). Furthermore the cell from which the genomic sample is prepared must present the same ploidy than the cell used for preparing the nucleic acid sample of the invention; i.e. the cells present the same chromosomal abnormalities (e.g. in case of cancer cells).

Combinations of pair of PCR primers that specifically amplify a sequence present in at least one copy per haploid genome and that is not transcribed to any significant extent at step f) and h) may be used.

All formula described in the methods of the invention may be calculated with adequate computer programs.

Another aspect of the invention relates to a pair of PCR primers, optionally combined with a sequence specific probe, pair of PCR primers that specifically amplify a sequence present in at least one copy per haploid genome (from any species such as mammals; e.g. human or mouse) and that is not transcribed to any significant extent. In some embodiments, the pair of PCR primers may amplify a sequence that may be present in at least one copy in the haploid genome.

In some embodiment said pair of PCR primers are selected in the Table A or B. Corresponding probes are also described in Table A.

Another aspect of the invention relates to a kit comprising a genomic DNA (gDNA) sample and at least one pair of PCR primers, optionally combined with a sequence specific probe, pair of PCR primers that specifically amplify a sequence present in at least one copy per haploid genome (from any species such as mammals; e.g. human or mouse) and that is not transcribed to any significant extent. In some embodiment said pair of PCR primers are selected in the Table A or B.

In some embodiment, the kit of the invention may further comprise a pair of PCR primers specific for a gene of interest. The kit according to the present invention may also contain a buffer. The kit may also contain an amount of deoxynucleoside triphosphates or deoxynucleotide triphosphates (also known as dNTP). Furthermore, such a kit according to the present invention may comprise a thermostable DNA polymerase such as Taq Polymerase, and all other reagents necessary for performing the amplification reaction, including but not limited to a buffer reagent, additional dNTP, and sequence specific amplification primers. In addition, the kit may comprise reagents necessary for detection of the amplicon during qPCR such as at least one fluorescently labeled hybridization probe, or a doubles stranded fluorescent dye.

The invention will be further illustrated by the following figures and examples. However, these examples and figures should not be interpreted in any way as limiting the scope of the present invention.

### FIGURES

FIG. 1: Correlation of ΔC_{q} RT(−)-RT(+) with percentage of gDNA

FIG. 2: Equivalence between CqDNA calculated by ValidPrime and RT(−) measurements

Fold ratios in linear scale (2^{-Cq}_{_{RT+}}/2^{-Cq}_{_{RT−}}) between either the total signal (NA) measured in spiked RT(+) reactions (dark bars) or the gDNA signal (DNA) estimated by ValidPrime (VP) from RT(+) reactions (light bars) compared to the signal in RT(−) reactions. Twenty nanograms of cDNA from adipose tissue (hatched bars) or from kidney, were spiked with 0.30 ng gDNA to decrease the variability due to stochastic amplification observed in RT(−) reactions. Independently of the expression level of the three genes studied in RT(+) samples, the estimations by ValidPrime of the gDNA-derived signals in RT(+) were very similar to the signals measured in RT(−) reactions, as the ratio was close to 1 (illustrated by the dashed line; mean 1.20+/−0.29). Data are mean +/−SD from two experiments in duplicate on the StepOnePlus.

FIG. 3: Correction of exogenous (spiked) gDNA with ValidPrime.

The data are presented in linear scale as fold ratio (2^{-Cq}/2^{Cq}_{_{ref}}), where Cq_{ref} is the Cq_{NA} measured on non-spiked controls and Cq refers to Cq_{RNA} (light bars) or Cq_{NA} (dark bars) depending on whether or not ValidPrime correction was applied (VP−/VP+). The data are grouped based on the impact of exogenous DNA, expressed as percentage of the total signal (%DNA) in each sample. Data were collected with either 17 GOI assays on a StepOnePlus (Applied Biosystems) using mVPA1 and mVPA5 (A), or with 19 assays on a BioMark (Fluidigm) using mVPA1 (B). All assays passed the high confidence ValidPrime criteria. Data are presented as the mean +/−SD, with (n) designating the number of samples in each group. cDNAs were from mouse kidney or liver for the StepOnePlus studies and mouse uterus for the BioMark study.

FIG. 4: ValidPrime applied on targets with one or multiple genomic loci.

cDNA samples were spiked with gDNA and analyzed with or without ValidPrime. Uncorrected ratios of relative quantities (left) and ValidPrime corrected ratios (right) are shown as mean +/−SD relative to the unspiked control. gDNA background contribution is indicated under the bars. (A) Data were generated with assays for Ch25h, Serpinel and Tgfb3, which all target a single locus. 88 samples were analyzed. (B) Using an assay targeting Gapdh, which has more than 50 well-conserved intronless pseudogenes in vertebrates and more than 300 in rodents (Liu et al., 2009), only 1 out of 25 samples spiked with up to 3000 genome copies had more than 60% DNA contribution as consequence of high Gapdh expression. Experiments were performed on the StepOnePlus.

FIG. 5: Capacity of mVPA1 to amplify low copy number templates. Determination of LOD and LOQ.

Different concentrations of gDNA (from 0 to 64 haploid genome copies) were used as template in qPCR reactions with the mVPA1 assay. Limit of detection (LOD) and PCR efficiency were determined with GenEx software and the limit of quantification (LOQ) was defined using SD<0.45 as the upper limit of precision. Taking the concentrations above LOD into account the PCR efficiency (E) was estimated to: 1.00+/−0.16 (95% CI). Similar results were obtained with the mVPA5 assay as well as with higher gDNA concentrations (see Table 6 and Material and Methods section).

### EXAMPLE 1: VALIDPRIME: QUANTIFYING AND CORRECTING QPCR FOR GENOMIC DNA WITHOUT DNASE

Quantitative real-time PCR has become a wide-spread method to monitor gene expression. Despite its straight-forward appearance, qPCR results can lead to incorrect interpretations for a variety of reasons. In response to the numerous caveats associated with qPCR experiments, the MIQE standards (Bustin et al., 2009), were developed to ensure that qPCR results are accurate, relevant and reproducible.

Due to the similar physicochemical properties of RNA and DNA, genomic DNA (gDNA) contamination is an inherent problem during RNA purification. The presence of gDNA can lead to non-specific amplification and aberrant result interpretation, since the contamination is not uniform and varies between samples (Bustin, 2002). The classical method to control for gDNA contamination involves the use of a RT- (RT-negative) sample. A difference of at least 6 cycles between reactions with and without RT implies that the gDNA derived signal derived is negligible (less than 3%). However, it has become commonplace to treat samples with DNase prior to reverse transcription to eliminate the role of DNA in its entirety.

We claim that DNase treatment is unnecessary for the majority of qPCR studies in eukaryotes, since it is possible to design gDNA-insensitive assays for most genes. Apart from adding extra costs to the protocol, the addition of the enzyme, buffers or added compounds, such as EDTA, can influence the performance of the qPCR. Incomplete DNAse inactivation can result in cDNA degradation post RT. Finally, DNase treatment does not alleviate the need for downstream controls that validate the absence of DNA-derived signal.

For these reasons, and also since gDNA removal in small sample preparations is difficult if not impossible, we have developed a procedure that measures gDNA contamination and evaluates its impact on the observed signal. We propose an equation that estimates the true RNA-derived signal by subtracting the gDNA-derived signal and demonstrate with proof-of-principle studies that efficient correction can be obtained even when gDNA contamination contributes to 50% of the total signal.

ValidPrime validates primers with regard to their sensitivity towards gDNA contamination. While it is possible to design assays where primer target sequences are separated by large introns (>0.8 kb) or utilize exon-exon junctions for the majority of eukaryotic genes (Roy and Gilbert, 2006), there remain a large number of cases where this strategy is not feasible. Examples include intronless genes and genes with conserved pseudogenes. Thus, it remains important to evaluate the impact of gDNA contamination.

Central to the ValidPrime method are the addition of gDNA controls to the experimental design. A gDNA specific assay (gDSA), (herein also referred to as ValidPrime assay (VPA) or gDNA assay) is used to assess the level of gDNA in the test samples [Cq(gDSA,test)] (see equation I).

Cq_{RT(-)}^{GOI}=Cq_{gDNA}^{GOI}+(Ca_{Sample}^{ValidPrime}−Cq_{gDNA}^{ValidPrime}) (I)

However, since the primers targeting the gene of interest (GOI) are likely to have a different ability to amplify gDNA than gDSA, the signal is normalized against an external reference gDNA (RefDNA) (herein also referred to as gDNA). RefDNA can be employed as a single sample or in a dilution series.

ValidPrime employs a different strategy when given data that include a RefDNA dilution series, which permits determination of the sensitivity of the GOI primers toward gDNA and calculation of the amplification efficiency. If the efficiencies of the GOI and the gDNA amplification of RefDNA are similar (+/−10%), a linear regression (LR) approach is possible as Cq(gDSA,test) becomes equal to Cq(gDSA,RefDNA). The LR method also evaluates the exact level of contamination in each sample, reported as the number of genomic copies. To simplify the analysis, we developed the ValidPrime software. The most basic functions of the method have also been integrated in the GenEx software.

Equation II enables the adjustment of the CqNA data (NA=nucleic acids, or DNA+RNA) and is based on the inherent difference between DNA and cDNA.

$$C {q}_{RNA}^{GOI} = - {log}_{2} ( {2}^{- {Cq}_{RT ( + )}^{GOI}} - {2}^{- {Cq}_{RT ( - )}^{GOI}} ) \qquad ( Equation II )$$

Whereas DNA supplies two strands that can serve as templates for PCR, the RNA moiety of the cDNA is not a substrate for the Taq polymerase. Hence, for a given quantity of starting material, RNA requires an additional cycle compared to DNA to reach the same amplification level.

As a first step to test the functionality of ValidPrime, gDNA assays were designed in intergenic regions devoid of any known transcriptional activity. Twenty-six out of thirty tested mouse gDNA assays displayed PCR efficiencies between 90-110% (97.5+/−4.3% (mean+/−SD)) on purified gDNA (50-5000 copies). Five assays were chosen for determination of LOD (limit of detection) and LOQ (limit of quantification) on RefDNA.

To verify that the PCR efficiency of the gDNA assay was unaffected in the presence of RNA or cDNA, different concentrations of RefDNA were spiked into total RNA and cDNA from liver and adipose tissue and into yeast tRNA. The overall efficiency was 99.1+/−2.1%, all 6 assay conditions comprised, indicating that the PCR efficiency of the gDNA assay was unaffected by the presence of RNA or cDNA.

In order to provide proof of principle, we chose 3 assays targeting single exons of 3 genes with a moderate expression level in mouse kidney: Ch25h (Cholesterol 25-hydroxylase), Serpine-1 (Plasminogen activator inhibitor-1) and Tgfb3 (Transforming growth factor beta-3), that amplified RefDNA with efficiencies between 95 and 105% (n=3/GOI). Using these 3 assays on mouse kidney cDNA, we compared the accuracy of ValidPrime regarding the determination of CqDNA (gDNA contamination level) with the classical RT-approach. The global RT-signal was 0.18+/−0.11% of the total signal, indicating a low level of DNA contamination and homogenous expression of the 3 genes. ValidPrime estimated the DNA level in the D-RT+ samples to 0.20+/−0.09% of the total signal. The RT− signal/CqDNA signal was 96.6+/−38.3% (n=3/GOI) providing support for a good correlation between the DNA level estimated by ValidPrime and the one measured in RT− samples. In order to circumvent the increased variability inherently associated with high Cq values frequently observed in RT− samples, we spiked both the RT− and D-RT+ samples with 100 copies of genomic DNA. Indeed, as expected, less dispersion was observed in the data and again a good correlation was observed (98.3+/−20.8%, n=2/GOI).

Taken together, these data show that ValidPrime accurately calculates the DNA-derived signal in non-Dnase treated samples, generating results similar to those obtained using RT− samples.

Next, we tested the capacity of ValidPrime to calculate CqRNA signals. GOI and gDNA signals were measured in both DNase (D+) and non-DNase treated (D−) RT+ samples that were spiked or not with increasing amounts of RefDNA. The added DNA contributed to up to 68% of the total signal. Overall, ValidPrime was able to correct for 96.2+/−8.2% of this signal. Similar results were obtained using either linear regression or the mean of CqRNAs generated with a RefDNA dilution series. Results are also consistent when only a single RefDNA concentration was used, or when the spiked samples were treated or not with DNAse. However, there was an occasional lack of precision in the correction when >60% of the signal was DNA-derived.

Similar analyses were performed with Gapdh, a highly expressed gene, which has a large number of retrotransposed pseudogenes. As shown in FIG. 4B, Validprime was able to correct for practically the entire spiked RefDNA-derived signal (99.5+/−0.8%) when <60% of the signal was DNA-derived.

Altogether, these data demonstrate that ValidPrime is able to distinguish and efficiently correct for DNA-derived signals, as long as they are not the predominant (>60%) signal source in the sample.

Even though it is technically possible to use RT− data as CqDNA input for the calculation of CqRNA, this implies multiplication of samples. Indeed, RT− controls are not well adapted for certain emerging high-throughput technologies, such as the Fluidigm/BioMark system, as it will use up to 50% of all available reaction chambers. The use of external RefDNA and gDNA assays reduce the number of control reactions by more than 85% compared to an RT- based approach.

In order to classify assays and samples, we propose a scoring scheme based on the relative levels of DNA derived signals. Assays that do not amplify gDNA are denoted A+assays (or ValidPrimers). Assays amplifying gDNA are graded A, B, C or F based on the level of contamination in each samples. This grading becomes especially useful when a large number of samples are studied, such as in the Fluidigm/Biomark technology. The experimental validation of the gDNA sensitivity is generally overseen in the characterization and quality-control of new qPCR assays. Validprime provides this possibility. We believe that the gDNA sensitivity (A+ vs. non-A+ assays) is an important criterion which should be taken into account in the global validation of qPCR assays. Validprime offers a simple, cost-efficient, universal tool for such analysis. The generation and experimental validation of A+ assays will ultimately decrease the need for DNase treatment of RNA prior to qPCR studies.

### EXAMPLE 2

ValidPrime™ is an assay to test for the presence of gDNA in test samples and when combined with a gDNA control sample, replaces all RT(−) controls. ValidPrime™ is highly optimized and specific to a non-transcribed locus of gDNA that is present in exactly one copy per haploid normal genome. Therefore, ValidPrime™ measures the number of genomic copies present in a sample and can be used for normalization of samples to cell copy number, as endogenous control for CNV applications, and as control for gDNA background in RTqPCR. The ValidPrime™ kit also contains a gDNA standard that can be used to test the sensitivity of RTqPCR assays for gDNA background. In expression profiling experiment the ValidPrime™ assay is added to the list of assays and the gDNA control is added to the list of samples. From the combined measurements with the ValidPrime™ assay and the gene of interest (GOI) assays on all samples and on the gDNA control the genomic background contribution to all RTqPCR measurements can be assessed. ValidPrime™ replaces the need to perform RT(−) controls for all reactions and makes RTqPCR profiling easier and substantially cheaper. In an expression profiling experiment based on m samples and n assays, conventional set up requires m RT(−) reactions followed by m×n qPCR controls, while using ValidPrime™ only m+n+1 controls are needed (Table 1).

TABLE 1
Number of control RT(−) and qPCR reactions needed to control for
gDNA background using traditional RT(−) approach and ValidPrime ™. ValidPrime
reduces the number of required controls in RT qPCR. ValidPrime replaces the need to
perform RT(−) controls for all RT(+) reactions and reduces substantially the number of
controls compared to a conventional set up. In an expression profiling experiment based on
m samples and n assays, the RT(−) approach requires m RT(−) reactions followed by m × n
qPCR controls, whereas ValidPrime only requires m + n + 1 controls. The numbers in the
table are based on single measurements for both approaches. Even when p gDNA
samples/concentrations are included in the experimental setup using ValidPrime, the
number of control reactions (m + (p × n) + p) is largely inferior to the RT(−) approach.

| No. of | | Assays (n) | | | | | | | | | |
|---|---|---|---|---|---|---|---|---|---|---|---|
| controls | | 1 | | 10 | | 24 | | 48 | | 96 | |
| Samples (m) | 1 | 2 | 3 | 11 | 12 | 25 | 26 | 49 | 50 | 97 | 98 |
| | 10 | 20 | 12 | 110 | 21 | 250 | 35 | 490 | 59 | 970 | 107 |
| | 24 | 48 | 26 | 264 | 35 | 600 | 49 | 1176 | 73 | 2328 | 121 |
| | 48 | 96 | 50 | 528 | 59 | 1200 | 73 | 2352 | 97 | 4656 | 145 |
| | 96 | 192 | 98 | 1056 | 107 | 2400 | 121 | 4704 | 145 | 9312 | 193 |
| | | RT− | VPA | RT− | VPA | RT− | VPA | RT− | VPA | RT− | VPA |

| | |
|---|---|
| traditional RT− strategy | ValidPrime ™ |
| (m × n) + m | (m + n + 1) |

Traditional Approach Based on RT(−) Controls

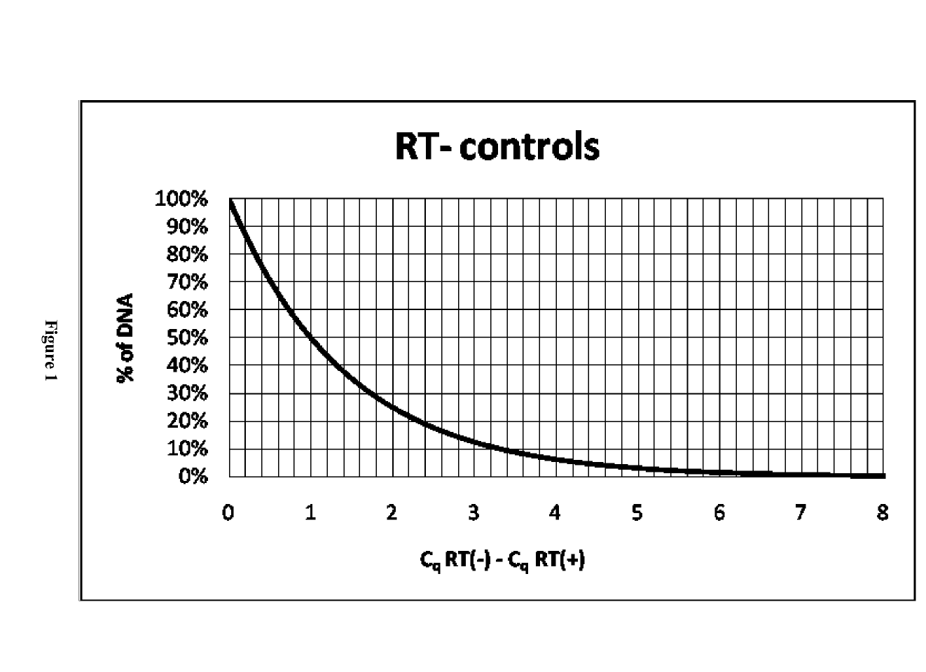

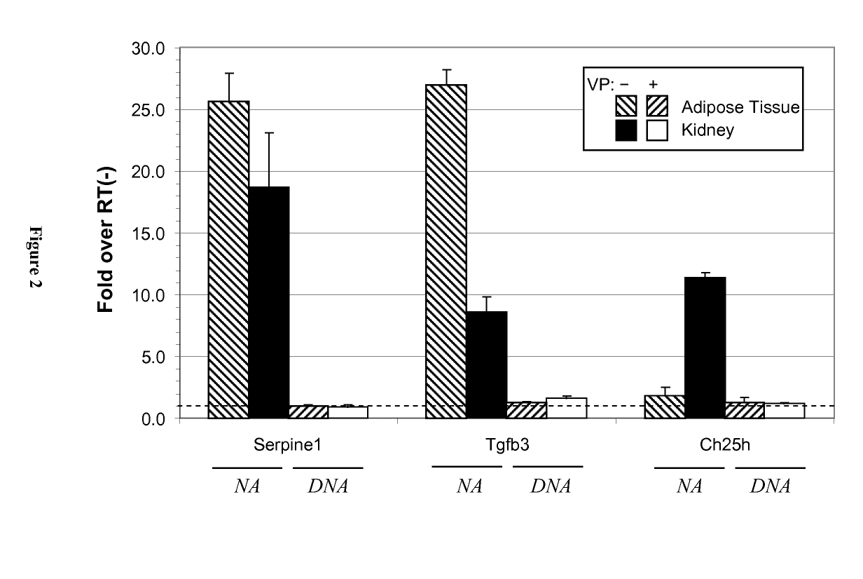

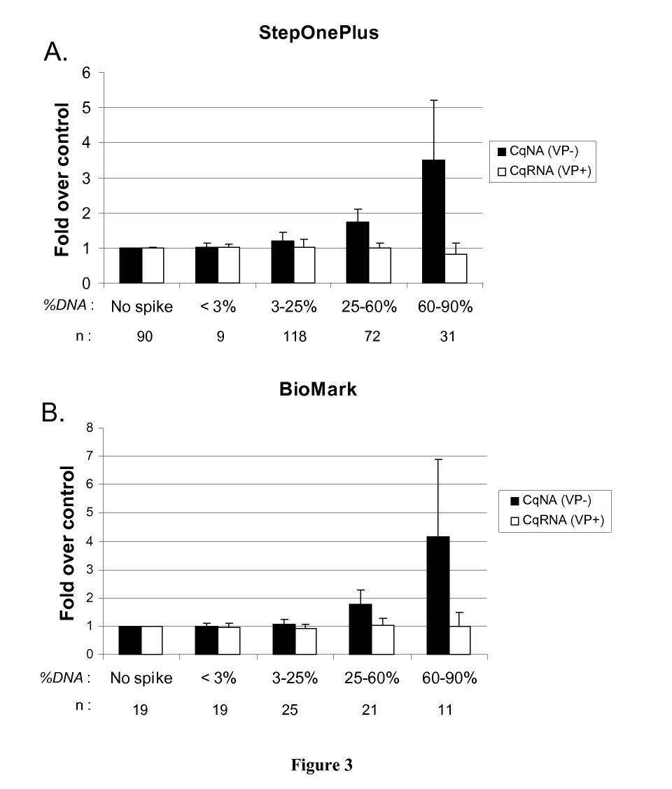

Presence of genomic background in RTqPCR expression profiling is conventionally assessed by running an RT(−) control for each sample that is analyzed by qPCR for all the GOI's. Any signal observed in these RT(−)qPCR's is due to presence of contaminating DNA that is amplified by the qPCR assay designed for GOI. A common criterion to accept the measured Cq as not being confounded by gDNA contamination is Cq_{RT}_{^{−}}^{GOI}−Cq_{RT}_{⁺}^{GOI}>5. The estimated GOI concentration is then accurate to at least 96.9% (FIG. 1).

If Cq_{RT}_{^{−Cq}}_{RT}_{⁺}^{GOI}<5 the measured Cq_{RT}_{^{−}}^{GOI} is confounded. It can be corrected to reflect the RNA concentration using eq. 1 (Laurell et al., 2012):

$$\begin{matrix}C {q}_{RNA}^{GOI} = - {log}_{2} ( {2}^{- {Cq}_{{RT}^{+}}^{GOI}} - {2}^{- {Cq}_{{RT}^{-}}^{GOI}} ) & \end{matrix} \qquad Equation 1$$

Cq_{RT}_{⁺}^{GOI} and Cq_{RT}_{^{−}}^{GOI} are the qPCR Cq values measured for the RT(+) and RT(−) reactions, and Cq_{RNA}^{GOI} is the Cq value that would have been obtained for the RT(+) reaction in absence of gDNA contaminations. From Cq_{RNA}^{GOI} the correct transcript amount can be calculated.

### ValidPrime™

Using ValidPrime™ the same test for gDNA contamination can be performed and, if needed, the same correction for background can be made, with a much smaller number of reactions. The sensitivities of the GOI qPCR assays for gDNA (Cq_{gDNA}^{GOI}) are tested relative to the ValidPrime™ assay (Cq_{gDNA}^{ValidPrime}) on the provided gDNA standard. Well performing GOI assays that have been properly designed to exclusively target mRNA, by, for example, having intron spanning primers shall not amplify the gDNA standard, while GOI assays that amplify sequences present in multiple copies in the gDNA will have even lower C_{q} values than the ValidPrime™ assay. All samples are then analyzed also with the ValidPrime™ assay (Cq_{Sample}^{ValidPrime}). The measurement setup is shown in Table 2.

TABLE 2
Experimental setup based on five samples assayed for four GOI's and
ValidPrime ™ including also the gDNA standard.

| Original data | GOI 1 | GOI 2 | GOI 3 | GOI 4 | ValidPrime |
|---|---|---|---|---|---|
| sample 1 | 20.1 Cq_{RT}_{⁺}^{GOI} | 31.1 Cq_{RT}_{⁺}^{GOI} | 22.1 Cq_{RT}_{⁺}^{GOI} | 28.2 Cq_{RT}_{⁺}^{GOI} | 32.5 Cq_{Sample}^{ValidPrime} |
| sample 2 | 20.5 Cq_{RT}_{⁺}^{GOI} | 31.2 Cq_{RT}_{⁺}^{GOI} | 22.5 Cq_{RT}_{⁺}^{GOI} | 28.9 Cq_{RT}_{⁺}^{GOI} | 33.2 Cq_{Sample}^{ValidPrime} |
| sample 3 | 21 Cq_{RT}_{⁺}^{GOI} | 31.1 Cq_{RT}_{⁺}^{GOI} | 22.9 Cq_{RT}_{⁺}^{GOI} | 30.2 Cq_{RT}_{⁺}^{GOI} | 32.3 Cq_{Sample}^{ValidPrime} |
| sample 4 | 23.1 Cq_{RT}_{⁺}^{GOI} | 31.8 Cq_{RT}_{⁺}^{GOI} | 22.5 Cq_{RT}_{⁺}^{GOI} | 32.3 Cq_{RT}_{⁺}^{GOI} | 34.2 Cq_{Sample}^{ValidPrime} |
| sample 5 | 23.5 Cq_{RT}_{⁺}^{GOI} | 30.8 Cq_{RT}_{⁺}^{GOI} | 22.8 Cq_{RT}_{⁺}^{GOI} | 32 Cq_{RT}_{⁺}^{GOI} | 33.1 Cq_{Sample}^{ValidPrime} |
| gDNA standard | 25.8 Cq_{gDNA}^{GOI} | 26.9 Cq_{gDNA}^{GOI} | 26.7 Cq_{gDNA}^{GOI} | 26 Cq_{gDNA}^{GOI} | 27 Cq_{gDNA}^{ValidPrime} |

Equation 2

Cq_{RT}_{^{−}}^{GOI}=Cq_{gDNA}^{GOI}+(Cq_{Sample}^{ValidPrime}−Cq_{gDNA}^{ValidPrime}) 2

From the measured Cq_{Sample}^{ValidPrime}, Cq_{gDNA}^{ValidPrime} and Cq_{gDNA}^{GOI} expected Cq for RT(−) controls, Cq_{RT}_{^{−}}^{GOI}, are calculated with Equation 2, and, as before, Equation 1 is used to correct for the gDNA background (Table 3).

TABLE 3
Measured Cq_{RT}⁺^{GOI} calculated Cq_{RT}⁻^{GOI} using Equation 2, and
calculated Cq_{RNA}^{GOI} using equation 1.

| | gene 1 | | | gene 2 | | |
|---|---|---|---|---|---|---|
| Validprime ™ | Cq_{RT}⁺^{GOI} | Cq_{RT}⁻^{GOI} | Cq_{RNA}^{GOI} | Cq_{RT}⁺^{GOI} | Cq_{RT}⁻^{GOI} | Cq_{RNA}^{GOI} |
| sample 1 | 20.1 | 31.3 | 20.10 | 31.1 | 32.4 | 31.85 |
| sample 2 | 20.5 | 32 | 20.50 | 31.2 | 33.1 | 31.65 |
| sample 3 | 21 | 31.1 | 21.00 | 31.1 | 32.2 | 32.01 |
| sample 4 | 23.1 | 33 | 23.10 | 31.8 | 34.1 | 32.13 |
| sample 5 | 23.5 | 31.9 | 23.50 | 30.8 | 33 | 31.15 |
| | gene 3 | | | gene 4 | | |
| Validprime ™ | Cq_{RT}⁺^{GOI} | Cq_{RT}⁻^{GOI} | Cq_{RNA}^{GOI} | Cq_{RT}⁺^{GOI} | Cq_{RT}⁻^{GOI} | Cq_{RNA}^{GOI} |
| sample 1 | 22.1 | 32.2 | 22.10 | 28.2 | 31.5 | 28.35 |
| sample 2 | 22.5 | 32.9 | 22.50 | 28.9 | 32.2 | 29.05 |
| sample 3 | 22.9 | 32 | 22.90 | 30.2 | 31.3 | 31.11 |
| sample 4 | 22.5 | 33.9 | 22.50 | 32.3 | 33.2 | 33.41 |
| sample 5 | 22.8 | 32.8 | 22.80 | 32 | 32.1 | 35.90 |

### EXAMPLE 3

### Correction of RT-qPCR Data for Genomic DNA-Derived Signals with ValidPrime

Material & Methods

Samples

All samples were from mouse (C57B1/6J) tissues (kidney, liver, adipose tissue, uterus, peritoneal macrophages). All experimental procedures involving animals were performed in accordance with the principles and guidelines established by the National Institute of Medical Research (INSERM) and were approved by the local Animal Care and Use Committee. Prior to sampling, mice were anesthetized by intraperitoneal injection of ketamine (100 mg kg⁻¹) and xylazine (10 mg kg⁻¹). Tissues were snap frozen in liquid nitrogen and stored at −80° C. Isolation of peritoneal macrophages has been described elsewhere (Calippe et al., 2008). Macrophages were in some cases treated with 20 ng/ml LPS ex vivo for 4 hours prior to RNA extraction.

DNA extraction

C57B1/6 mouse genomic DNA was extracted from whole blood using the PerfectPure DNA Blood Cell Kit, according to the recommended protocol (5′PRIME GmbH, Hamburg, Germany). Good results were also obtained with gDNA purified from mouse tails by phenol/chloroform extraction after Proteinase K digestion (Hofstetter et al., 1997). The DNA concentration was determined spectroscopically (NanoDrop).

RNA extraction

Total RNA was extracted using a double purification protocol. Briefly, Trireagent (Sigma-Aldrich, Saint Louis, Mich.) was added to the frozen tissue sample, which was homogenized in a Precellys 24 homogenizer (Bertin Technologies, France). After the extraction step the supernatant was gently mixed with one volume 70% ethanol and applied on a total RNA miniprep Genelute column, where it was washed and eluted following the instructions from the manufacturer (Sigma-Aldrich). The integrity and quality of the RNA was tested by capillary micro-electrophoresis (MultiNA (Shimadzu) or Experion (BioRad)) and spectroscopically (NanoDrop). A fraction of the RNA was DNase treated using the DNAfree kit from Ambion. To avoid inhibition of the reverse transcriptase, the volume of DNAse treated RNA did not exceed 25% of the total volume during reverse transcription.

Reverse Transcription

Total RNA (1.0-5.0 μg) was reverse transcribed in 20-50 μL using the High Capacity cDNA Reverse Transcription Kit (Applied Biosystems) using random hexamers. The reaction mixture was incubated for 10 min at 25° C., 120 min at 37° C., and finally for 5 min at 85° C., according to instructions from the manufacturer (Applied Biosystems). RT reactions were diluted 5-10 fold prior to qPCR.

Real-time qPCR

Conventional qPCR. All reactions (except when indicated) were performed in duplicate 10 μL volumes using 20 ng reverse transcribed total RNA in a StepOnePlus system (Applied Biosystems) with the SsoFast EvaGreen Supermix (BioRad) and an assay concentration of 300 nM using the cycling parameters: 95° C. (20 sec) followed by 40 cycles at 95° C. (3 sec) and 60° C. (20 sec). Melting curve analysis: 95° C. (15 sec); 60° C. (60 sec) and a progressive increase up to 95° C. (0.5° C./min). Analysis of the data was performed with the StepOne software v.2.2.

High throughput qPCR. 96.96 Dynamic Arrays for the microfluidic Biomark system (Fluidigm Corporation, Calif., USA) (Spurgeon et al., 2008) were used to study gene expression in 6.5 ng cDNA from mouse peritoneal macrophages or mouse uterus, as described below.

Specific Target Amplification. Pre-amplification of cDNA (produced from 25-65 ng of total RNA) was performed in the StepOnePlus cycler (Applied Biosystems) (95° C.-10min activation step followed by 14 cycles: 95° C., (15sec), 60° C., (4min)) in a total volume of 5 pl in the presence of all primers at a concentration of 50 nM. After pre-amplification, 20 μL Low EDTA TE Buffer (10 mM Tris pH8 (Ambion), 0.1 mM EDTA pH8 (Sigma)) was added to each sample.

Sample Mix for Biomark analysis. The pre-sample mix contained 66.7% 2×Taqman® Gene Expression Master Mix (Applied Biosystems), 6.67% 20× DNA Binding Dye Sample Loading Reagent (Fluidigm), 6.67% 20× EvaGreen™ (Biotium), 20% Low EDTA TE Buffer. Sample mix was obtained by mixing 5.6 μL of the pre-sample mix with 1.9 μL of diluted cDNA.

Assay Mix. 3.8 μL 2× Assay Loading Reagent (Fluidigm) and 1.9 μL Low EDTA TE Buffer were mixed with 1.9 μL of primers (20 μM of each forward and reverse primer). qPCR conditions. After priming of the 96.96 Dynamic Array in the NanoFlex™ 4-IFC Controller (Fluidigm), 5 μL of each sample and 5 μL of each assay mix were added to dedicated wells. The dynamic array was then placed again in the IFC Controller for loading and mixing under the following conditions: 50° C., (2 min); 70° C., (30 min) and 25° C. (10 min). The loaded Dynamic Array was transferred to the BioMark™ real-time PCR instrument. After initial incubation at 50° C., (2 min) and activation of the Hotstart enzyme at 95° C., (10 min) cycling was performed using 95° C., (15sec), and 60° C. (1 min) for 35 cycles, followed by melting curve analysis (1° C./3sec).

Data analysis. Initial data analysis was performed with the Fluidigm real-time PCR analysis software v. 3.0.2 with linear derivative baseline correction and a quality correction set to 0.65.

Design of ValidPrime Assays (VPA)

Intergenic regions in the mouse genome with no known transcriptional activity were selected using the UCSC genome browser (http://genome.ucsc.edu/). Thirty assays targeting 10 different regions on 5 chromosomes were designed using PrimerBlast (NCBI). Amplification efficiencies were determined with a dilution series of gDNA (50 to 5000 haploid genome copies). PCR products were analyzed for purity by recording melting curves and by capillary micro-electrophoresis (MultiNA, Shimadzu), leading to the selection of 5 assays for LOD and LOQ determination.

LOD and LOQ Determination of VPAs

Five assays were selected for determination of LOD (limit of detection) and LOQ (limit of quantification) using 8 concentrations (0, 1, 2, 4, 8, 16, 32, 64 copies) in the presence of 50 ng/μl carrier yeast tRNA (Roche Molecular Biochemicals). Sequence information for the 2 best candidates, in terms of sensitivity and specificity, is provided in Table 4. Except when stated otherwise, mVPA1 was used as the VPA.

GOI Assay Design and Validation

Non-commercial GOI assays were either taken from previously published studies (Calippe et al 2008, Riant et al 2009, Giulietti et al 2001) or designed with the Primer-BLAST utility at NCBI. Sequences are reported in Table 4. Specificity was evaluated by BLAST (mouse RefSeq database) during design and by in silico PCR (UCSC Genome Browser). Amplification efficiencies were evaluated in the BioMark system on dilutions series of both cDNA and gDNA.

Exogenous gDNA Spiking Experiments

Quantities ranging from 50 to 5000 haploid genome copies (corresponding to 0.15-15 ng gDNA) or water were added to 20 ng (StepOnePlus) or 6.5 ng (BioMark) cDNA. Non-spiked samples had low, but detectable gDNA levels. For the BioMark runs, the gDNA was added prior to the pre-amplification step. Genome copy number calculations were based on the NCBI m37 assembly of the C57B1/6 mouse genome (2,716,965,481 bp) assuming an average molecular weight of 660 g/mol per base pair. The mass of a haploid mouse genome was thus estimated to be 2.98 pg.

Data Analysis and Statistics

Cq_{DNA}, Cq_{RNA} and % DNA were calculated using the gh-validprime software (https://code.google.com/p/gh-validprime). The GenEx software (v.5.3, www.multiD.se) was used for one-way ANOVA analysis and to calculate LOD. Data are presented as mean +/−SD.

TABLE 4
Primer sequence information for GOI assays and VPAs. A+ indicates
primers that do not amplify gDNA. UCSC references refer to sequence coordinates in the
genome browser at UCSC (http://genome.ucsc.edu) according to the NCBI37/mm9 assembly.

| | | | | |
|---|---|---|---|---|
| GOI | | | Amplicon | |
| Symbol | Forward | Reverse | length | UCSC Gene Reference |

High confidence GOI assays used in the StepOneplus qPCR system

| Ccl2 | Ccl2 Forward | Ccl2 Reverse | 80 | uc007kmp.1_CcI2: 418 + 497 |
|---|---|---|---|---|
| Cebpa | Cebpa Forward | Cebpa Reverse | 87 | uc009gjl.1_Cebpa: 1712 + 1798 |
| Ch25h | Ch25h Forward | Ch25h Reverse | 78 | uc008hgj.1_Ch25h: 622 + 699 |
| Fdx1 | Fdx1 Forward | Fdx1 Reverse | 79 | uc009plo.1_Fdx1: 256 + 334 |
| Gapdh | Included in the mouse reference gene panel (Tataa biocenter) | | | |
| Tataa | | | | |
| Plat | Plat Forward | Plat Reverse | 93 | uc009Idx.2_Plat: 900 + 992 |
| Ppia | Ppia Forward | Ppia Reverse | 125 | uc007hyn.1_Ppia: 108 + 232 |
| Ppia | Included in the mouse reference gene panel (Tataa biocenter) | | | |
| Tataa | | | | |
| Ptgs1 | Ptgs1 Forward | Ptgs1 Reverse | 70 | uc008jll.1_Ptgs1: 266 + 335 |
| Retnla | Retnla Forward | Retnla Reverse | 80 | uc007zjy.1_Retnla: 221 + 300 |
| Rhoa | Rhoa Forward | Rhoa Reverse | 125 | uc009rpe.2_Rhoa: 353 + 477 |
| Serpine1 | Serpine1 Forward | Serpine1 Reverse | 75 | uc009abn.2_Serpine1: 1229 + 1303 |
| Vcam1 | Vcam1 Forward | Vcam1 Reverse | 106 | uc008rbx.1_Vcam1: 564 + 669 |

High confidence GOI assays used in the Biomark system

| | | | | |
|---|---|---|---|---|
| Cebpb | Cebpb Forward | Cebpb Reverse | 72 | uc008oaf.1_Cebpb: 1210 + 1281 |
| Ctsf | Ctsf Forward | Ctsf Reverse | 112 | uc008gbc.1_Ctsf: 883 + 994 |
| Cyr61 | Cyr61 Forward | Cyr61 Reverse | 113 | uc008rqq.2_Cyr61: 336 + 448 |
| Id1 | Id1 Forward | Id1 Reverse | 95 | uc008ngf.1_Id1: 134 + 228 |
| Ier2 | Ier2 Forward | Ier2 Reverse | 90 | uc009mmr.2_Ier2: 739 + 828 |
| Igfbp5 | Igfbp5 Forward | Igfbp5 Reverse | 79 | uc007bkx.1_Igfbp5: 1402 + 1480 |
| Ipo5 | Ipo5 Forward | Ipo5 Reverse | 84 | uc007uzz.1_Ipo5: 33 + 116 |
| Junb | Junb Forward | Junb Reverse | 101 | uc012ghn.1_Junb: 1138 + 1 |
| Mad2l1 | Mad2l1 Forward | Mad2l1 Reverse | 120 | uc009cen.1_Mad2l1: 1096 + 1215 |
| Nfkbia | Nfkbia Forward | Nfkbia Reverse | 84 | uc007nor.2_Nfkbia: 574 + 657 |
| Pik3r2 | Pik3r2 Forward | Pik3r2 Reverse | 105 | uc009mbn.2_Pik3r2: 796 + 900 |
| Rasd1 | Rasd1 Forward | Rasd1 Reverse | 90 | uc007jfh.1_Rasd1: 634 + 723 |
| Sox4 | Sox4 Forward | Sox4 Reverse | 74 | uc007pyk.1_Sox4: 1407 + 1480 |
| Sprr2f | Sprr2f Forward | Sprr2f Reverse | 82 | uc008qdu.1_Sprr2f: 86 + 167 |
| Ubb | Ubb Forward | Ubb Reverse | 77 | uc007jjg.1_Ubb: 254 + 330 |

High confidence GOI assays used in both qPCR systems

| | | | | |
|---|---|---|---|---|
| Gapdh | Gapdh Forward | Gapdh Reverse | 123 | uc009dts.1_Gapdh: 58 + 180 |
| Mapk3 | Mapk3 Forward | Mapk3 Reverse | 144 | uc009jsm.1_Mapk3: 343 + 486 |
| Odc1 | Odc1 Forward | Odc1 Reverse | 108 | uc007ncv.1_Odc1: 343 + 450 |
| Tgfb3 | Tgfb3 Forward | Tgfb3 Reverse | 69 | uc007ohn.2_Tgfb3: 1672 + 1741 |

GOI assays used in the Biomark for correction of endogenous DNA

| | | | | |
|---|---|---|---|---|
| Il1b | Il1b gDNA A + Forward | Il1b gDNA A+ Reverse | 152 | uc008mht.1_Il1b: 527 + 678 |
| gDNA | | | | |
| A+ | | | | |
| Il1b | Il1b gDNA Forward | Il1b gDNA Reverse | 50 | uc008mht.1_Il1b: 359 + 408 |
| gDNA | | | | |
| Serpine1 | Serpine1 A+ Forward | Serpine1 A+ Reverse | 71 | uc009abn.2_Serpine1: 1133 + 1203 |
| A+ | | | | |
| Serpine1 | Serpine1 Forward | Serpine1 Reverse | 75 | uc009abn.2_Serpine1: 1229 + 1303 |
| Chi313 | Chi3l3 A+ Forward | Chi3l3 A+ Reverse | 96 | uc008qvw.2_Chi3l3: 480 + 575 |
| A+ | | | | |
| Chi3l3 | Chi3l3 Forward | Chi3l3 Reverse | 109 | uc008qvw.2_Chi3l3: 154 + 262 |

VPAs used in the study

| | | | | |
|---|---|---|---|---|
| mVPA1 | GGAGCCCAGTGTAGAAGAGCA | AGCCAGCGAACCATATCCTGA | 87 | chr1:41857082 + 41857168 |
| | SEQ ID NO: 10 | SEQ ID NO: 11 | | |
| mVPA5 | ACAGGAGAGCCACGTGTATCC | ACTCCCTGTTCTTGACGTGCT | 111 | chr5:117408738 + 117408848 |
| | SEQ ID NO: 12 | SEQ ID NO: 13 | | |

Results

The ValidPrime Method

We developed ValidPrime to estimate and correct for gDNA contribution in RT(+)-qPCR measurements in a more reliable manner than that afforded by RT(−) controls. We refer to the signal measured in an RT(+)-qPCR as Cq_{NA} (NA:Nucleic Acids) (Equation 1), indicating contributions from RNA (Cq_{RNA}) as well as gDNA (Cq_{DNA}) as shown in Equation 2, expressed in relative quantities. GOI refers to any transcribed “gene-of-interest”, including reference genes, studied in a RT-qPCR experiment.

$$\begin{matrix}{Cq}_{RT +}^{GOI} = {Cq}_{NA}^{GOI} & ( 1 ) \\ {2}^{- {Cq}_{NA}^{GOI}} = {2}^{- {Cq}_{RNA}^{GOI}} + {2}^{- {Cq}_{DNA}^{GOI}} & ( 2 )\end{matrix}$$

Traditionally, determination of the RNA component using RT(−) controls would be achieved using Equation 3. However, as detailed in the introduction, low reproducibility and other factors detract from the accuracy of this approach. We propose that Equation 4, provides an accurate solution provided that Cq_{DNA} is estimated using ValidPrime (Equation 5). Cq_{RNA} and Cq_{DNA} refer to the signal contribution derived from RNA (cDNA) and DNA (gDNA), respectively, in a RT+ sample.

$$\begin{matrix}{Cq}_{RNA}^{GOI} = - {log}_{2} ( {2}^{- {Cq}_{RT +}^{GOI}} - {2}^{- {Cq}_{RT -}^{GOI}} ) & ( 3 ) \\ {Cq}_{RNA}^{GOI} = - {log}_{2} ( {2}^{- {Cq}_{NA}^{GOI}} - {2}^{- {Cq}_{DNA}^{GOI}} ) & ( 4 ) \\ {Cq}_{DNA}^{GOI} = {Cq}_{Sample}^{VPA} + {Cq}_{gDNA}^{GOI} - {Cq}_{gDNA}^{VPA} & ( 5 )\end{matrix}$$

For the determination of Cq_{DNA} (Equation 5), the gDNA contamination level in a RT(+) sample (referred to as “Sample”) is measured with a gDNA-specific ValidPrime Assay (VPA) (Cq_{Sample}^{VPA}). The VPA targets a non-transcribed locus present in one copy per normal haploid genome. However, since the gDNA sensitivity can be highly variable between GOI assays, the capacity of the GOI assay to amplify gDNA is compared with that of the VPA. In ValidPrime, this difference is tested on purified gDNA, yielding the delta Cq component in Equation 5 (Cq_{gDNA}^{GOI}−Cq_{gDNA}^{VPA}). Despite a formulaic resemblance to the ΔΔCt equation developed by Livak and Schmittgen, 2001, these calculations are distinct.

Table 5 depicts a typical grid of qPCR data including the required controls for ValidPrime estimation of Cq_{DNA} and the subsequent correction of Cq_{NA} into Cq_{RNA}. Apart from the GOI assays, that are specific for each study, the VPA has been added among the assays. In addition to samples 1-3, which correspond to any RT+ samples in qPCR study, one or several gDNA samples are added in the experimental design. The equations under the grid exemplify the calculations for GOI 1 in Sample 1. The gDNA contribution can also be expressed as a percentage of relative quantities (Equation 6).

| | |
|---|---|
| $\% DNA = ( {2}^{- {Cq}_{DNA}} / {2}^{- {Cq}_{NA}} ) * 100$ | (6) |
| ${2}^{- {Cq}_{NA}} = {2}^{- {Cq}_{RNA}} + {2}^{- {Cq}_{DNA}}$ | (Eq 2) |

| Cq_{NA} | GOI 1 | GOI 2 | GOI 3 | VPA |
|---|---|---|---|---|
| Sample 1 | 27.22 | 25.78 | 28.67 | 29.02 |
| Sample 2 | 26.73 | 25.54 | 28.02 | 26.97 |
| Sample 3 | 26.42 | 25.31 | 27.68 | 26.34 |
| gDNA | 29.62 | 29.41 | 30.60 | 28.61 |

| | |
|---|---|
| $\begin{matrix}{Cq}_{DNA} = {Cq}_{Sample}^{VPA} + {Cq}_{gDNA}^{GOI} - {Cq}_{gDNA}^{VPA} \\ {Cq}_{{DNA}_{S 1}^{GOI 1}} = 29.02 + 29.62 - 28.61 = 30.03\end{matrix}$ | (Eq 5) |
| $\begin{matrix}{Cq}_{RNA} = - {log}_{2} ( {2}^{- {Cq}_{NA}} - {2}^{- {Cq}_{DNA}} ) \\ {Cq}_{{NA}_{S 1}^{GOI 1}} = 27.22 \\ {Cq}_{{RNA}_{S 1}^{GOI 1}} = - {log}_{2} ( {2}^{- 27.22} - {2}^{- 30.03} ) = 27.44\end{matrix}$ | (Eq 3) |
| $\begin{matrix}\% DNA = ( {2}^{- {Cq}_{DNA}} / {2}^{- {Cq}_{NA}} ) * 100 \\ \% {DNA}_{S 1}^{GOI 1} = ( {2}^{- 30.03} / {2}^{- 27.22} ) * 100 = 14.3 \%\end{matrix}$ | (Eq 6) |

ValidPrime uses the annotation Cq_{NA} for the signal measured in a (RT+) qPCR sample, to which both Nucleic Acids, RNA and DNA contribute, corresponding to Cq_{RNA} and Cq_{DNA} (Eq. 2). The grid shows an example of an experimental design with 3 RT+ samples and 3 GOI assays, plus the controls required for the ValidPrime estimation of Cq_{DNA} and the subsequent correction of Cq_{NA} to obtain Cq_{RNA}. The term GOI is used in ValidPrime for both target transcripts and reference genes, since the calculations are independent of the gene type. The VPA column contains the data obtained with the ValidPrime Assay and the gDNA row contains measurements using purified genomic DNA as a sample. The equations under the grid illustrate the determination of Cq_{DNA}, Cq_{RNA} and % DNA for GOI 1 in sample 1.

Assay Validation

In order to determine the accuracy of the ValidPrime method, we first designed and characterized candidate VPAs. Among 30 candidates from ten different regions on five chromosomes, 26 amplified gDNA with efficiencies between 90-110%. Among the tested assays, mVPA1 (amplifying an 87 by sequence in the qB region of chromosome 1) and mVPA5 (amplifying an 87 by sequence in the qF region of chromosome 5) had the best characteristics in terms of sensitivity and specificity. LOD was 3.2 copies for mVPA1 (GenEx; Cut-off Cq 37; 95% CI; mean of 2 determinations) and 3.7 copies for mVPA5 (GenEx; Cut-off Cq 37; 95% CI) and the LOQ (SD<45%) was 4 copies for both assays (FIG. 5). In 4 out of 8 NTC reactions a signal (Cq 38.1+/−0.9) was detected with the mVPA5 assay, indicating formation of primer-dimers. However, the primer-dimer product was never observed in samples containing gDNA, as evaluated by melting curve analyses and by capillary micro-electrophoresis (MultiNA, Shimadzu).

Efficiency analysis for GOI assays was performed in the BioMark system. No amplification was observed in the NTC controls except for Sprr2f (Cq 28.6), which was 10 cycles above the Cq measured in the sample with the lowest Sprr2f expression (Cq 18.5) and thus far more than the proposed accepted minimal difference of 5 cycles between NTC and RT(+) sample (11,12). The generally low Cq values obtained with the BioMark system are explained by the 14 cycle preamplification step used in this protocol. The amplification efficiency was similar between assays as measured with a cDNA dilution series (95.5+/−6.1%; mean R²:0.9932) and a gDNA dilution series for gDNA-sensitive assays (100.4+/−7.7%; mean R²:0.9962). All RNA samples used in the study had A260/A280 ratios between 1.9-2.0 (mean: 1.97); A260/A230 between 1.5-2.5 (mean: 2.13) and A260/A270 above 1.17 (mean: 1.23), where the latter tests for phenol contamination.

TABLE 6
Presence of cDNA or RNA does not affect the quantification of gDNA
with VPA. Addition of 20 ng of cDNA (DNase treated), RNA (DNase treated) or tRNA to
different amounts of gDNA (0.15, 0.6, 3.0 ng corresponding to 50, 200, 1000 haploid genome
copies) did not affect the amplification efficiency (expressed in %) of mVPA1
(n = 4/condition). A one-way ANOVA analysis (Tukey-Kramer, GenEx v.5.3) did not reveal
any significant difference between the groups with same gDNA concentration. Indeed, the
coefficient of variation (CV), calculated on relative quantities (2^{-Cq}), of all 24 data points
(=6 * 4) for each gDNA concentration was less than 20%. Furthermore; the absence of signal
(ND) in all samples with no gDNA demonstrates that there is no transcriptional activity at the
VPA locus.

| Spiking | | gDNA | | SD | | VPA | |
|---|---|---|---|---|---|---|---|
| Source | Description | copy# | | (n = 4) | slope | Efficiency (%) | R² |
| | | | Mean Cq | | | | |
| | | | (VPA) | | | | |
| Adipose | DNase treated | 0 | ND | | | | |
| Tissue | cDNA | 50 | 30.46 | 0.02 | −3.267 | 102.3 | 0.9997 |
| | | 200 | 28.55 | 0.18 | | | |
| | | 1000 | 26.21 | 0.06 | | | |
| Adipose | DNase treated | 0 | ND | | | | |
| Tissue | RNA | 50 | 30.42 | 0.36 | −3.365 | 98.2 | 0.9999 |
| | | 200 | 28.43 | 0.25 | | | |
| | | 1000 | 26.05 | 0.08 | | | |
| Liver | DNase treated | 0 | ND | | | | |
| | cDNA | 50 | 30.33 | 0.06 | −3.378 | 97.7 | 0.9994 |
| | | 200 | 28.39 | 0.07 | | | |
| | | 1000 | 25.94 | 0.09 | | | |
| Liver | DNase treated | 0 | ND | | | | |
| | RNA | 50 | 30.51 | 0.23 | −3.397 | 96.9 | 0.9993 |
| | | 200 | 28.57 | 0.12 | | | |
| | | 1000 | 26.1 | 0.07 | | | |
| Yeast | tRNA | 0 | ND | | | | |
| | | 50 | 30.26 | 0.34 | −3.303 | 100.8 | 0.9982 |
| | | 200 | 28.1 | 0.09 | | | |
| | | 1000 | 25.95 | 0.05 | | | |
| H₂O | H₂O | 0 | ND | | | | |
| | | 50 | 30.26 | 0.28 | −3.359 | 98.5 | 0.9978 |
| | | 200 | 28.42 | 0.18 | | | |
| | | 1000 | 25.9 | 0.06 | | | |
| | | | | | Mean | 99.2 | 0.9993 |
| | | | | | SD | 2.3 | 0.0007 |
| | | | CV (%) | | | | |
| | | | (n = 24) | | | | |
| | | 50 | 16.26 | | | | |
| | | 200 | 14.52 | | | | |
| | | 1000 | 8.51 | | | | |

Equivalence Between C_{DNA} Estimated with ValidPrime and RT(−) Controls

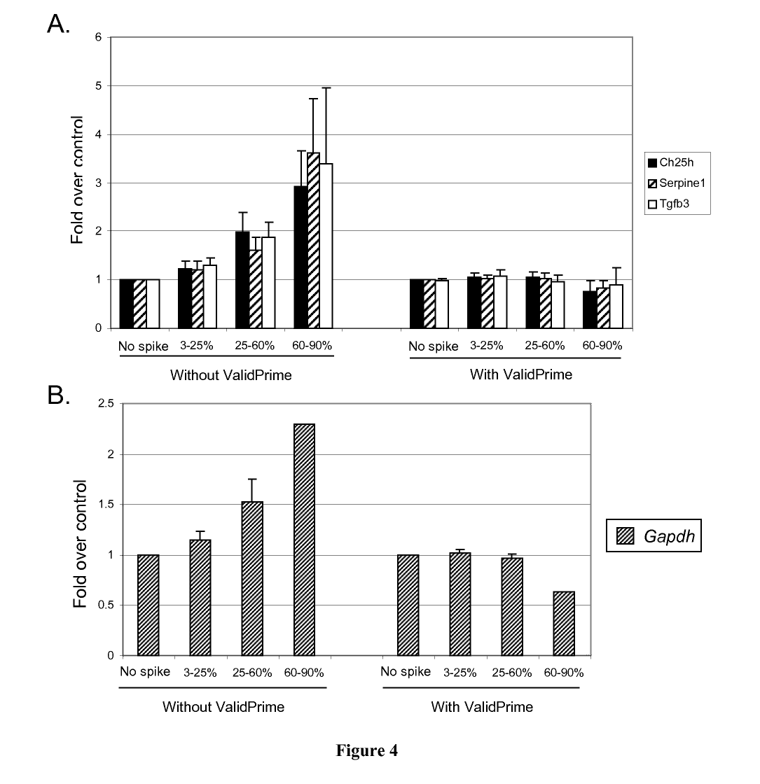

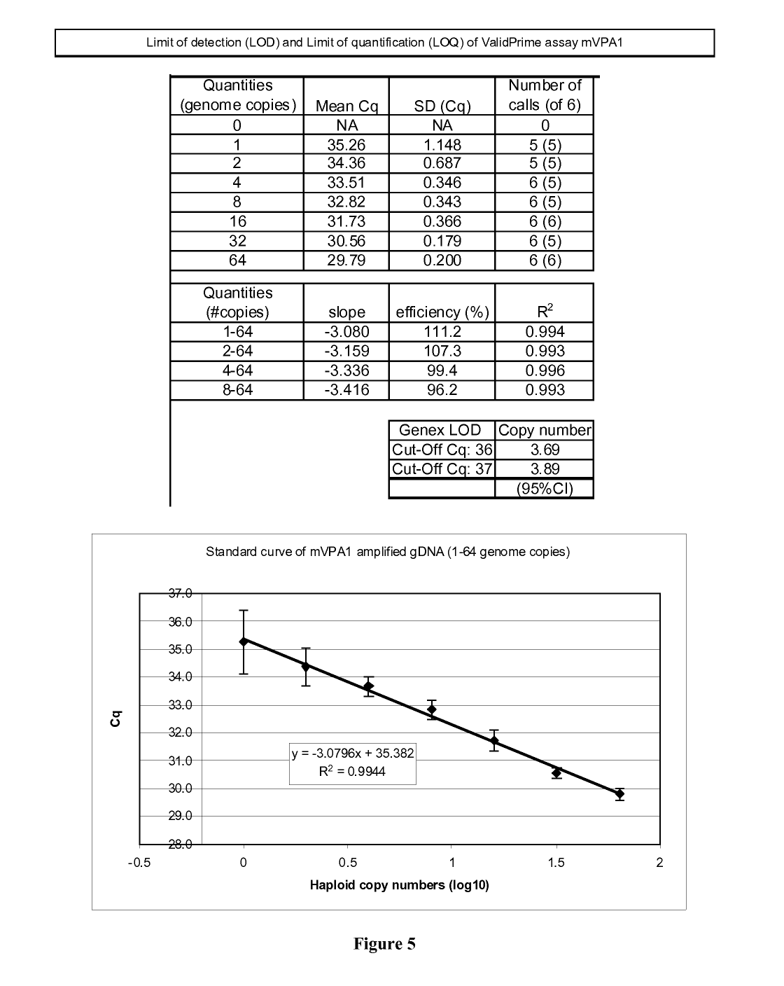

We next verified that the Cq_{DNA} values calculated with ValidPrime agree with those measured directly in RT(−)-qPCRs. Since a direct comparison is difficult, due to the poor reproducibility of RT(−) controls (see above), the following test was performed. RT(+) and RT(−) samples from 2 different tissues were spiked with 0.30 ng of gDNA (2100 haploid genome copies) and measured using three gDNA-sensitive GOI assays. The data in FIG. 2 are ratios of relative quantities (RQ) between either the total signal (Cq_{NA}) in RT(+) reactions or the corresponding Cq_{DNA} calculated by ValidPrime over the RQ in RT(−) reactions. As shown, tissue-dependent differences in the expression levels of the three target genes were observed (from 1.8 to 27 fold compared to RT(−) samples). Independent of the expression level, the estimation by ValidPrime of the gDNA-derived signal levels (Cq_{DNA}) in RT(+) samples was in excellent agreement with the data from RT(−) samples, with the ratio of the relative quantities (1.20+/−0.29) close to the theoretically expected value of 1.

Calculation of Cq_{RNA} in RT(+) Samples Through the Correction of Signals Derived from Exogenously Added gDNA

Given the good correlation between ValidPrime estimation of Cq_{DNA} and RT(−) measurements, we next tested the accuracy of the calculation of the RNA-derived component Cq_{RNA} in RT(+) samples using Equation 4. In a first set of experiments, different amounts of gDNA were added to cDNA test samples with low, but detectable, endogenous gDNA levels. All 32 GOI assays were gDNA-sensitive (Table 4) and had gDNA amplification efficiencies similar to the VPA (i.e. passed the ValidPrime high confidence criteria). Both the traditional StepOnePlus microtiter plate based qPCR (FIG. 3A) and the microfluidic BioMark system (Spurgeon et al., 2008) (FIG. 3B) were used to collect raw data (Cq_{NA}) as input for ValidPrime estimations of the RNA-derived signal (Cq_{RNA}). Samples were grouped according to the level of DNA contribution. Using ValidPrime we could accurately estimate the RNA-derived signal (Cq_{RNA}) even in samples with elevated gDNA levels. However, the correction was less precise when the gDNA background exceeded 60% of the total signal. The demonstration that with ValidPrime we can identify and correct for signals derived from exogenous DNA in experimental RT-qPCR samples, using two different qPCR platforms, was first step towards a “proof-of-principle”. The correction is virtually independent of gene copy number since it works well both for GOI assays targeting one single locus and for genes with multiple pseudogenes (FIG. 4).

Correction of Signals Derived from Endogenous gDNA

In order to evaluate the capacity of ValidPrime to correct for endogenous gDNA present in typical RNA preparations, a different strategy was applied. We used a gDNA-sensitive and a gDNA-insensitive assay for each GOI, with comparable amplification efficiencies. Three genes (Il1b, Serpine1 and Chi3l3) expressed in mouse macrophages were chosen as targets. Using the BioMark system, qPCR data were collected from 81 RNA preparations and the ValidPrime correction was applied. Despite identical overall gDNA content, the impact of the gDNA on the total signal obtained with the gDNA-sensitive assays differed considerably between the three genes. When the impact was limited (i.e. low % DNA), as in the case for Il1b, the effect of the ValidPrime correction was modest. With increasing % DNA, as observed for Serpine1 and Chi3l3, the result of the correction becomes clearer, even in log2 scale. Theoretically, given identical amplification efficiencies for the 2 assays and the absence of gDNA amplification, the Cq_{NA} data should fall on a straight line with a slope of 1. The presence of gDNA will contribute to the signal measured with gDNA sensitive assays (x-axis), and the uncorrected Cq_{NA} data will therefore produce a slope>1. Even though the impact of the correction differs for the three genes, the Cq_{RNA} values estimated using ValidPrime restore linearity, especially for samples with a DNA contribution below 60%.

These data demonstrate that using ValidPrime, efficient correction of RT-qPCR data for the presence of endogenous gDNA is possible as long as the DNA contribution to the total signal is less than 60%.

Discussion

Since its invention in the early/mid nineties (Higuchi et al., 1993; Gibson et al., 1996) qPCR has undergone considerable methodological and technological advances (Pfaffl, 2010). However, despite its direct impact on qPCR results, no alternative to RT(−) controls has, to our knowledge, been proposed to assess gDNA-derived contributions to the signals in RT-qPCR.

ValidPrime is a cost-efficient alternative to RT(−) controls to test for the presence of gDNA in samples. It is superior to RT(−) controls not only because of a higher accuracy but also because fewer control reactions are required, eliminating the need for additional test reactions in the RT step. While the traditional approach for a study based on m samples and n genes requires m reverse transcription control reactions (RT−) and m×n extra qPCRs, ValidPrime only requires m+n+1 control qPCRs and no RT (−) reactions (Table 1). As an example, in a BioMark 96.96 Dynamic Array experiment, ValidPrime reduces the number of controls by more than 95%.

ValidPrime is also the first method that proposes to correct for qPCR signals originating from contaminating gDNA. It is possible that the lack of accuracy and low reproducibility generally observed in RT(−) reactions has previously restrained the development of a correction-based model similar to that proposed in Equation 3. The present study includes data obtained with cDNA from 5 different mouse tissues analyzed with two qPCR instrument platforms, providing support for the general validity of ValidPrime.

It is important not to confuse gDNA contamination levels with the actual contribution of gDNA to the total signal, herein expressed as % DNA (Equation 6). Indeed, we did not observe any correlation between gDNA levels (as estimated by qPCR with the VPA) and the total signal (Cq_{NA}) measured in RT(+) qPCR reactions with GOI assays. However, there is a clear positive correlation between % DNA and Cq_{NA} with the gDNA sensitive assay, which demonstrates the increased impact of contaminating gDNA in samples with low GOI expression levels.

The primer design strategy also strongly influences the impact of gDNA on the qPCR signal. Given the multi-exonic nature of most eukaryotic genes (Roy and Gilbert, 2006), it is conceivable that gDNA-insensitive assays can be designed for most targets in vertebrates. Regardless of the primer design strategy, the inability of a GOI assay to amplify gDNA needs to be validated experimentally. ValidPrime offers this possibility. However, for certain targets it is impossible to design transcript-specific assays. This can be due to either the presence of intronless pseudogenes or the absence of introns in single-exon genes. In order to assure a good accuracy for the ValidPrime correction, these gDNA sensitive assays should behave similarly to VPA against gDNA. In analogy with the comparative Ct method (or AACt method) (Livak and Schmittgen, 2001), in which similar amplification efficiencies for the GOI and reference gene assays are presumed, estimation of Cq_{RNA} in ValidPrime assumes similar efficiencies for the GOI and gDNA assays.

When validated according to the MIQE guidelines (Bustin et al., 2009), gDNA-sensitive assays are in general perfectly compatible with ValidPrime. Nevertheless, when using a GOI assay for the first time with ValidPrime, and especially when Cq adjustment is requested, we recommend the inclusion of a gDNA dilution series with concentrations covering at least 3 log₁₀ (eg. 5-5000 haploid genomic copies). Consistent relation to VPA across the dilution series indicates similar amplification efficiencies of the two assays, which sanctions Cq correction with high confidence. For VPAs, as well as for high confidence GOI assays, we generally observed perfectly linear amplifications from 5 to 10,000 haploid genomic copies (corresponding to 0.015-30 ng) (FIG. 5). Even though it is possible that higher gDNA concentrations (i.e. >30 ng/reaction) could influence qPCR amplification efficiencies (Yun et al., 2006), such gDNA contamination levels are rarely, if ever, encountered in RT-qPCR experiments. Furthermore, we did not observe any differences in the VPA amplification between samples with purified gDNA and mixed samples, spiked with cDNA or RNA (Table 6).

Even though we consistently observed very low variability between replicates in VPA-gDNA amplifications over a wide range of initial gDNA concentrations (FIG. 5 and Table 6), it is advisable to use 1-10 ng gDNA (i.e. ≈300-3000 haploid genome copies) per qPCR, when only one gDNA concentration is included in the design. This range favors reliable and distinct gDNA amplification with the VPA and the “high confidence” gDNA-sensitive GOI assays. It also increases the confidence when verifying the absence of gDNA amplification with GOI assays that are presumed to be “gDNA-insensitive”.

In this study we used a maximal standard deviation of 0.3 for the ΔCq between VPA and GOI gDNA amplifications as criterion for high confidence gDNA-sensitive GOI assays. Alternatively, an efficiency (E) based criterion can be used. Indeed, similar results to those shown in FIG. 3, were obtained when a maximal difference of 0.15 in E (defined as 10^{-1/slope}−1) was used as inclusion criterion. If a gDNA-sensitive GOI assay has a sub-optimal, but confidently estimated E and cannot be replaced with a better assay, Equation 7 (Kubista et al., 2007), or equivalent (Pfaffl, 2001), can be used to correct the Cq_{NA}. Procedures for confident determination of amplification efficiencies are described elsewhere (Tholen et al., 2003).

Cq_{NAnew}=Cq_{NAold}(log(1+E)/log(2)) (7)

Coherency of PCR product melting curve profiles from cDNA and gDNA samples should also be considered prior to Cq_{RNA} calculations. If a GOI assay generates gDNA-specific products that are not observed in cDNA samples, Cq_{RNA} adjustment of Cq_{NA} will not be reliable and is not recommended or even needed. Electrophoresis-based analysis of PCR-products is an alternative informative tool to verify that the same products are formed.

Caution should also be taken if differences in ploidy are expected, such as in cancer biopsies, since the number of VPA and GOI targets per cell could vary between samples. However, homogenous populations of aneuploid samples can be analysed with ValidPrime, such as cancer cell lines, given that the VPA and GOI target loci are each present at least in one copy per cell.

To make ValidPrime readily available, we have developed a software application (gh-validprime). ValidPrime Cq_{RNA} calculation is also available within the data pre-processing workflow of the GenEx software (version 5.3, www.multid.se). The gh-validprime software assigns grades to assays/samples based on the impact of the genomic background. gDNA-insensitive assays are classified as A+. Other assays are attributed the grades A, B, C, and F, where the assignment is sample-dependent. While A (<3% DNA) does not require correction, B and C samples (3-25 and 25-60% DNA, respectively) are corrected provided the assays pass the high confidence criteria. If gDNA contribution exceeds 60%, correction is not recommended. RT+ samples with gDNA concentrations below the limit of detection, in which the VPA fail to generate a signal, are attributed the grade A*. The default output from the ValidPrime software is either Cq_{NA} (for A+ assays, A* and A samples), Cq_{RNA} (for B and C samples) or “HIGHDNA” for F samples. The output data are ready for further pre-processing, such as normalization against reference genes. The gDNA sensitivity and confidence evaluation of GOI assays can be performed independently, or together with RT(+) samples, which facilitates the specificity assessment.

The ValidPrime source code is available through the gh-validprime project at https://code.google.com/p/gh-validprime. This software depends on the Qt framework (http://qt.nokia.com) and the GeneHuggers library (https://code.google.com/p/genehuggers). A windows installer and test files are available at http://code.google.com/p/gh-validprime/downloads/list.

ValidPrime assays targeting different species (including human, mouse and a general vertebrate) have been developed by the TATAA Biocenter (www.tataa.com).

ValidPrime provides, for the first time, the opportunity to correct reliably for gDNA background in qPCR. Correction is possible for any GOI assay that consistently amplifies gDNA, given that the DNA contribution does not exceed 60% of the signal. ValidPrime is superior to traditional RT(−) controls because of its higher accuracy and the lower number of controls required, which leads to a substantial cost savings.

TABLE A

| | Name of | | | | |
|---|---|---|---|---|---|
| Gene | assay | Forward | Reverse | length | refseq |
| mouse | M_15qE3 | TAGTGTGGTAAAGCAGAAAC | GGAACAATGACTTAGGGACA | 105 | chr15: 93,484,127- |
| | | SEQ ID NO: 1 | SEQ ID NO: 2 | | 93,495,021 |
| human | H_10q23.1 | AGCACATTTCTATTCTCCGT | TCTTGACCTTCTCTACCTCC | 143 | chr10: 86,510,623- |
| | | SEQ ID NO: 3 | SEQ ID NO: 4 | | 86,516,336 |
| universal | U_gUCR294 | GATTCAAAAAGTCCAGTCCC | TAGTATCTCCCCACCAAAA | 142 | Chr10: 102,037,760 |
| | | SEQ ID NO: 5 | SEQ ID NO: 6 | | −102,039,091 |
| mouse | M_15qE3 | TTATACAAGTTGGCACCATG | | | |
| | | TCACCGC | | | |
| | | SEQ ID NO: 7 | | | |
| human | H_10q23.1 | CCTGTGTAGGATGGTCCTGTT | | | |
| | | CCAATACCT | | | |
| | | SEQ ID NO: 8 | | | |
| universal | U_gUCR294 | TGCAGAACAGTCCTCATAA | | | |
| | | CTCATCCGA | | | |
| | | SEQ ID NO: 9 | | | |

TABLE B

| | Name of | | | | |
|---|---|---|---|---|---|
| Gene | assay | Forward | Reverse | length | refseq |
| mouse | m1qB_2 | GGAGCCCAGTGTAGAAGAGCA | AGCCAGCGAACCATATCCTGA | 87 | chr1: 41857082- |
| | | SEQ ID NO: 10 | SEQ ID NO: 11 | | 41857168 |
| mouse | m5qF_2 | ACAGGAGAGCCACGTGTATCC | ACTCCCTGTTCTTGACGTGCT | 111 | chr5: 117408738- |
| | | SEQ ID NO: 12 | SEQ ID NO: 13 | | 117408848 |
| mouse | m19qB_2 | CAGGGTTCATACCATCCTGGGT | AAGCCCTGCACTTCTCCATCA | 133 | chr19: 20394622- |
| | | SEQ ID NO: 14 | SEQ ID NO: 15 | | 20394754 |
| mouse | m19qC3_2 | CACCAGTCTGAACAACACGCA | GGCTCGGTGAACCAAATCCAA | 108 | chr19: 38441761- |
| | | SEQ ID NO: 16 | SEQ ID NO: 17 | | 38441868 |

### REFERENCES

Throughout this application, various references describe the state of the art to which this invention pertains. The disclosures of these references are hereby incorporated by reference into the present disclosure.

Bengtsson, M., Hemberg, M., Rorsman, P. and Stahlberg, A. (2008) Quantification of mRNA in single cells and modelling of RT-qPCR induced noise. BMC Mol Biol, 9, 63.

Bustin, S. A. (2002) Quantification of mRNA using real-time reverse transcription PCR (RT-PCR): trends and problems. J. Mol. Endocrinol., 29, 23-39.

Bustin, S. A., Benes, V., Garson, J. A., Hellemans, J., Huggett, J., Kubista, M., Mueller, R., Nolan, T., Pfaffl, M. W., Shipley, G. L. et al. (2009) The MIQE guidelines: minimum information for publication of quantitative real-time PCR experiments. Clin. Chem., 55, 611-622.

Calippe, B., Douin-Echinard, V., Laffargue, M., Laurell, H., Rana-Poussine, V., Pipy, B., Guery, J. C., Bayard, F., Arnal, J. F. and Gourdy, P. (2008) Chronic estradiol administration in vivo promotes the proinflammatory response of macrophages to TLR4 activation: involvement of the phosphatidylinositol 3-kinase pathway. J. Immunol., 180, 7980-7988.

Gibson, U. E., Heid, C. A. and Williams, P. M. (1996) A novel method for real time quantitative RT-PCR. Genome Res., 6, 995-1001.

Giulietti, A., Overbergh, L., Valckx, D., Decallonne, B., Bouillon, R. and Mathieu, C. (2001) An overview of real-time quantitative PCR: applications to quantify cytokine gene expression. Methods, 25, 386-401.

Higuchi, R., Fockler, C., Dollinger, G. and Watson, R. (1993) Kinetic PCR analysis: real-time monitoring of DNA amplification reactions. Biotechnology. (N.Y). 11, 1026-1030.

Hofstetter, J. R., Zhang, A., Mayeda, A. R., Guscar, T., Nurnberger, J. I., Jr. and Lahiri, D. K. (1997) Genomic DNA from mice: a comparison of recovery methods and tissue sources. Biochem. Mol. Med., 62, 197-202.

Kubista, M., Sindelka, R., Tichopad, A., Bergkvist, A., Lindh, D. and Forootan, A. (2007) The Prime Technique. Real-time PCR data analysis. G.I.T. Laboratory Journal, 9-10, 33-35.

Laurell, H., Iacovoni, J., Abot, A., Svec, D., Maoret, J. J., Arnal, J. F. and Kubista, M. (2012) Correction of RTqPCR data for genomic DNA-derived signals with ValidPrime. Nucleic Acids Res. (in press)

Liu, Y. J., Zheng, D., Balasubramanian, S., Carriero, N., Khurana, E., Robilotto, R. and Gerstein, M. B. (2009) Comprehensive analysis of the pseudogenes of glycolytic enzymes in vertebrates: the anomalously high number of GAPDH pseudogenes highlights a recent burst of retrotrans-positional activity. BMC Genomics, 10, 480.

Livak, K. J. and Schmittgen, T. D. (2001) Analysis of relative gene expression data using real-time quantitative PCR and the 2(-Delta Delta C(T)) Method. Methods, 25, 402-408.

Nordg\dot{a}rd, O., Kvaloy, J. T., Farmen, R. K. and Heikkila, R. (2006) Error propagation in relative real-time reverse transcription polymerase chain reaction quantification models: the balance between accuracy and precision. Anal. Biochem., 356, 182-193.

Peccoud, J. and Jacob, C. (1996) Theoretical uncertainty of measurements using quantitative polymerase chain reaction. Biophys. J., 71, 101-108.

Pfaffl, M. W. (2010) The ongoing evolution of qPCR. Methods, 50, 215-216.

Pfaffl, M. W. (2001) A new mathematical model for relative quantification in real-time RT-PCR. Nucleic Acids Res, 29, e45.

Riant, E., Waget, A., Cogo, H., Arnal, J. F., Burcelin, R. and Gourdy, P. (2009) Estrogens protect against high-fat diet-induced insulin resistance and glucose intolerance in mice. Endocrinology, 150, 2109-2117.

Roy, S. W. and Gilbert, W. (2006) The evolution of spliceosomal introns: patterns, puzzles and progress. Nat Rev Genet, 7, 211-221.

Spurgeon, S. L., Jones, R. C. and Ramakrishnan, R. (2008) High throughput gene expression measurement with real time PCR in a microfluidic dynamic array. PLoS One, 3, e1662.

Tholen, D. W., Kroll, M., Astles, J. R., Caffo, A. L., Happe, T. M., Krouwer, J. and Lasky, F. (2003) Evaluation of the linearity of quantitative measurement procedures: a statistical approach; approved guideline. CLSI EP6-A. Clinical and Laboratory Standards Institute, Wayne, Pa., 23 (16), 1-60.

Yun, J. J., Heisler, L. E., Hwang, II, Wilkins, O., Lau, S. K., Hyrcza, M., Jayabalasingham, B., Jin, J., McLaurin, J., Tsao, M. S. et al. (2006) Genomic DNA functions as a universal external standard in quantitative real-time PCR. Nucleic Acids Res, 34, e85.

1. A method for determining the expression level of a gene of interest (GOI) in a nucleic acid sample by means of reverse transcription real-time PCR comprising the steps of:
a) providing two aliquots of the nucleic acid sample
b) providing a pair of PCR primers specific for the gene of interest
c) treating a first aliquot with reverse transcriptase to produce complementary DNA (cDNA), performing a PCR on said aliquot with said pair of PCR primers of step b) and determining the Cq value (Cq_{RT(+)}^{GOI})
d) performing a PCR on a second aliquot of the nucleic acid sample with said pair of PCR primers of step b) and determining the Cq value (Cq_{RT(-)}^{GOI})
e) determining the expression level of said gene of interest (GOI) (Cq_{RNA}^{GOI}) with the formula
$$C {q}_{RNA}^{GOI} = - {log}_{2} ( {2}^{- {Cq}_{RT ( + )}^{GOI}} - {2}^{- {Cq}_{RT ( - )}^{GOI}} ) .$$

2. A method for determining the expression level of a gene of interest (GOI) in a nucleic acid sample by means of reverse transcription real-time PCR comprising the steps of:
a) providing a genomic DNA (gDNA) sample
b) providing a pair of PCR primers specific for the gene of interest
c) providing of at least one pair of PCR primers that specifically amplify a sequence present in at least one copy per haploid genome and that is not transcribed to any significant extent
d) treating said nucleic acid sample with reverse transcriptase to produce complementary DNA (cDNA)
e) performing a quantitative PCR on said nucleic acid sample of step d) with said pair of PCR primers of step b) and determining the Cq value (Cq_{RT(+)}^{GOI})
f) performing a quantitative PCR on said nucleic acid sample of step d) with said pair of PCR primers of step c) and determining the Cq value (Cq_{Sample}^{ValidPrime})
g) performing a quantitative PCR on the genomic DNA (gDNA) sample with said pair of PCR primers of step b) and determining the Cq value (Cq_{gDNA}^{GOI})
h) performing a quantitative PCR on the genomic DNA (gDNA) sample with the pair of PCR primers of step c) and determining the Cq value (Cq_{gDNA}^{ValidPrime})
i) calculating the Cq_{DNA}^{GOI} with the formula Cq_{DNA}^{GOI}=Cq_{gDNA}^{GOI}+(Cq_{Sample}^{ValidPrime}−Cq_{gDNA}^{ValidPrime})
j) determining the expression level of said gene of interest (GOI) (Cq_{RNA}^{GOI}) with the formula
$$C {q}_{RNA}^{GOI} = - {log}_{2} ( {2}^{- {Cq}_{RT ( + )}^{GOI}} - {2}^{- {Cq}_{DNA}^{GOI}} )$$

3. A method for determining the expression level of a gene of interest (GOI) in a nucleic acid sample by means of reverse transcription real-time PCR comprising the steps of:
a) providing two aliquots of the nucleic acid sample
b) providing two aliquots of a genomic DNA (gDNA) sample
c) providing a pair of PCR primers specific for the gene of interest
d) providing of at least one pair of PCR primers that specifically amplify a sequence present in at least one copy per haploid genome and that is not transcribed to any significant extent
e) treating a first aliquot of the nucleic acid sample with reverse transcriptase to produce complementary DNA (cDNA), performing a quantitative PCR on said aliquot with said pair of PCR primers of step c) and determining the Cq value (Cq_{RT(+)}^{GOI})
f) performing a quantitative PCR on the second aliquot of the nucleic acid sample with said pair of PCR primers of step d) and determining the Cq value (Cq_{Sample}^{ValidPrime})
g) performing a quantitative PCR on the first aliquot of the genomic DNA (gDNA) sample with said pair of PCR primers of step c) and determining the Cq value (Cq_{gDNA}^{GOI})
h) performing a quantitative PCR on the second aliquot of the genomic DNA (gDNA) sample with the pair of PCR primers of step d) and determining the Cq value (Cq_{gDNA}^{ValidPrime})
i) calculating the with the formula Cq_{DNA}^{GOI}=Cq_{gDNA}^{GOI}+(Cq_{Sample}^{ValidPrime}−Cq_{gDNA}^{ValidPrime})
j) determining the expression level of said gene of interest (GOI) (Cq_{RNA}^{GOI}) with the formula
$$C {q}_{RNA}^{GOI} = - {log}_{2} ( {2}^{- {Cq}_{RT ( + )}^{GOI}} - {2}^{- {Cq}_{DNA}^{GOI}} )$$

4. The method according to claim 3 wherein the quantitative PCR of step f) is performed on the second aliquot of the nucleic acid sample that was previously reverse transcribed using reverse transcriptase to produce complementary DNA (cDNA).

5. A kit for determining the expression level of a gene of interest (GOI) in a nucleic acid sample by means of reverse transcription real-time PCR comprising at least one pair of PCR primers that specifically amplify a sequence present in at least one copy per haploid genome and that is not transcribed to any significant extent and that is optionally combined with a sequence specific probe.

6. The kit according to claim 5 which further comprises a genomic DNA (gDNA) sample.

7. The kit according to claim 6 which further comprises a pair of PCR primers specific for a gene of interest.

8. The kit according to claim 5 which further comprises a buffer, an amount of deoxynucleoside triphosphates or deoxynucleotide triphosphates, a thermostable DNA polymerase, and reagents necessary for detection of the amplicon during qPCR.

9. A nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

10. The kit of claim 8, wherein said reagents include at least one fluorescently labeled hybridization probe and a double-stranded DNA binding fluorescent dye.

11. The method according to claim 2 wherein said pair of PCR primers that specifically amplify a sequence present in at least one copy per haploid genome and that is not transcribed to any significant extent are selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 10 and SEQ ID NO: 11, SEQ ID NO: 12 and SEQ ID NO: 13, SEQ ID NO: 14 and SEQ ID NO: 15, SEQ ID NO: 16 and SEQ ID NO: 17.

12. The method according to claim 3 wherein said pair of PCR primers that specifically amplify a sequence present in at least one copy per haploid genome and that is not transcribed to any significant extent are selected from the group consisting of SEQ ID NO: land SEQ ID NO: 2, SEQ ID NO: 3 and SEQ ID NO: 4, SEQ ID NO: 5 and SEQ ID NO: 6, SEQ ID NO: 10 and SEQ ID NO: 11, SEQ ID NO: 12 and SEQ ID NO: 13, SEQ ID NO: 14 and SEQ ID NO: 15, SEQ ID NO: 16 and SEQ ID NO: 17.

13. The kit according to claim 5 wherein said pair of PCR primers that specifically amplify a sequence present in at least one copy per haploid genome and that is not transcribed to any significant extent are selected from the group consisting of SEQ ID NO: land SEQ ID NO: 2, SEQ ID NO: 3 and SEQ ID NO: 4, SEQ ID NO: 5 and SEQ ID NO: 6, SEQ ID NO: 10 and SEQ ID NO: 11, SEQ ID NO: 12 and SEQ ID NO: 13, SEQ ID NO: 14 and SEQ ID NO: 15, SEQ ID NO: 16 and SEQ ID NO: 17.


SEQUENCE LISTING (17 sequences)

SEQ ID NO: 1 - DNA, 20 bp, Artificial
  Synthetic oligonucleotide primer
tagtgtggtaaagcagaaac

SEQ ID NO: 2 - DNA, 20 bp, Artificial
  Synthetic oligonucleotide primer
ggaacaatgacttagggaca

SEQ ID NO: 3 - DNA, 20 bp, Artificial
  Synthetic oligonucleotide primer
agcacatttctattctccgt

SEQ ID NO: 4 - DNA, 20 bp, Artificial
  Synthetic oligonucleotide primer
tcttgaccttctctacctcc

SEQ ID NO: 5 - DNA, 20 bp, Artificial
  Synthetic oligonucleotide primer
gattcaaaaagtccagtccc

SEQ ID NO: 6 - DNA, 19 bp, Artificial
  Synthetic oligonucleotide primer
tagtatctccccaccaaaa

SEQ ID NO: 7 - DNA, 27 bp, Artificial
  Synthetic oligonucleotide probe
ttatacaagttggcaccatgtcaccgc

SEQ ID NO: 8 - DNA, 30 bp, Artificial
  Synthetic oligonucleotide probe
cctgtgtaggatggtcctgttccaatacct

SEQ ID NO: 9 - DNA, 28 bp, Artificial
  Synthetic oligonucleotide probe
tgcagaacagtcctcataactcatccga

SEQ ID NO: 10 - DNA, 21 bp, Artificial
  Synthetic oligonucleotide primer
ggagcccagtgtagaagagca

SEQ ID NO: 11 - DNA, 21 bp, Artificial
  Synthetic oligonucleotide primer
agccagcgaaccatatcctga

SEQ ID NO: 12 - DNA, 21 bp, Artificial
  Synthetic oligonucleotide primer
acaggagagccacgtgtatcc

SEQ ID NO: 13 - DNA, 21 bp, Artificial
  Synthetic oligonucleotide primer
actccctgttcttgacgtgct

SEQ ID NO: 14 - DNA, 22 bp, Artificial
  Synthetic oligonucleotide primer
cagggttcataccatcctgggt

SEQ ID NO: 15 - DNA, 21 bp, Artificial
  Synthetic oligonucleotide primer
aagccctgcacttctccatca

SEQ ID NO: 16 - DNA, 21 bp, Artificial
  Synthetic oligonucleotide primer
caccagtctgaacaacacgca

SEQ ID NO: 17 - DNA, 21 bp, Artificial
  Synthetic oligonucleotide primer
ggctcggtgaaccaaatccaa